SEQUENCE LISTING (36 sequences)

SEQ ID NO: 1 - protein, 5 aa, artificial
  Synthesized IgG hinge region sequence
CPPCP

SEQ ID NO: 2 - protein, 9 aa, artificial
  Synthesized L3
GSGGSGGSG

SEQ ID NO: 3 - protein, 11 aa, artificial
  Synthesized L4
GGSGGSGGSGG

SEQ ID NO: 4 - protein, 14 aa, artificial
  Synthesized Linker
GGSGGSGGSGGSGG

SEQ ID NO: 5 - protein, 16 aa, artificial
  Synthesized L5a
GSGGSGGSGGSGGSGG

SEQ ID NO: 6 - protein, 16 aa, artificial
  Synthesized L5
GGSGGSGGSGGSGGSG

SEQ ID NO: 7 - protein, 20 aa, artificial
  Synthesized L6
GGSGGSGGSGGSGGSGGSGG

SEQ ID NO: 8 - protein, 261 aa, artificial
  Synthesized ANPxy-L4-Fc1ab (protein)
SLRRSSCFGGRMDRIGAQSGLGCNSFRYGGSGGSGGSGGCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 270 aa, artificial
  Synthesized ANPxy-L6-Fc1ab (protein)
SLRRSSCFGGRMDRIGAQSGLGCNSFRYGGSGGSGGSGGSGGSGGSGGCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 260 aa, artificial [sequence omitted]

SEQ ID NO: 11 - protein, 269 aa, artificial
  Synthesized ANPxy-L6-Fc2ab
SLRRSSCFGGRMDRIGAQSGLGCNSFRYGGSGGSGGSGGSGGSGGSGGCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 251 aa, artificial
  Synthesized ANPxy-L2-Fc1ab (protein)
SLRRSSCFGGRMDRIGAQSGLGCNSFRYGGCPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 260 aa, artificial [sequence omitted]

SEQ ID NO: 14 - protein, 222 aa, artificial
  Synthesized Fc1AB
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 28 aa, artificial
  Synthesized hANP28
SLRRSSCFGGRMDRIGAQSGLGCNSFRY

SEQ ID NO: 16 - protein, 32 aa, artificial
  Synthesized hBNP32
SPKMVQGSGCFGRKMDRISSSSGLGCKVLRRH

SEQ ID NO: 17 - protein, 221 aa, artificial
  Synthesized Fc2AB
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - protein, 239 aa, artificial
  Synthesized CysFc-004
METDTLLLWVLLLWVPGSTGCPPCPAPELLGGPSVFLFPPKPKDTLMISRTPEVTCVVVDSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCVKGFYPSDIAVEWESNGQPENNYKTTPPVLDSDGSFFLYSKLTVDKSRWQQGNFSCSVMHEALHNHYTQKSLSLSPGK

SEQ ID NO: 19 - protein, 30 aa, artificial
  Synthesized Peptide A
SLRRSSCFGGRMDRIGAQSGLGCNSFRYGG

SEQ ID NO: 20 - protein, 39 aa, artificial
  Synthesized Peptide B
SLRRSSCFGGRMDRIGAQSGLGCNSFRYGGSGGSGGSGG

SEQ ID NO: 21 - protein, 44 aa, artificial
  Synthesized Peptide C
SLRRSSCFGGRMDRIGAQSGLGCNSFRYGSGGSGGSGGSGGSGG

SEQ ID NO: 22 - protein, 28 aa, artificial
  Synthesized Peptide D
SLRRSSCFGGRMDRIGAQSGLGCNSFRY

SEQ ID NO: 23 - protein, 37 aa, artificial
  Synthesized Peptide E
GSGGSGGSGSLRRSSCFGGRMDRIGAQSGLGCNSFRY

SEQ ID NO: 24 - DNA, 843 bp, artificial
  Synthesized ANPxy-L4-Fc1ab (nucleotide) + signal sequence
atggagacagacacactcctgctatgggtactgctgctctgggttccaggttccactggtagcctgcggagatccagctgcttcgggggcaggatggacaggattggagcccagagcggactgggctgtaacagcttccggtacggcggatccggcgggtccggcggatccggcggatgcccaccgtgcccagctccggaactgctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 25 - protein, 281 aa, artificial
  Synthesized ANPxy-L4-Fc1ab (protein) + signal sequence
METDTLLLWVLLLWVPGSTGSLRRSSCFGGRMDRIGAQSGLGCNSFRYGGSGGSGGSGGCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 870 bp, artificial
  Synthesized ANPxy-L6-Fc1ab (nucleotide) + signal sequence
atggagacagacacactcctgctatgggtactgctgctctgggttccaggttccactggtagcctgcggagatccagctgcttcgggggcaggatggacaggattggagcccagagcggactgggctgtaacagcttccggtacggcggatccggcgggtccggcggctccggcggctccggcgggtccggcggatccggcggatgcccaccgtgcccagctccggaactgctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 27 - protein, 290 aa, artificial
  Synthesized ANPxy-L6-Fc1ab (protein) + signal sequence
METDTLLLWVLLLWVPGSTGSLRRSSCFGGRMDRIGAQSGLGCNSFRYGGSGGSGGSGGSGGSGGSGGCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 840 bp, artificial
  Synthesized ANPxy-L4-Fc2ab (nucleotide) + signal sequence
atggagacagacacactcctgctatgggtactgctgctctgggttccaggttccactggtagcctgcggagatccagctgcttcgggggcaggatggacaggattggagcccagagcggactgggctgtaacagcttccggtacggcggatccggcgggtccggcggatccggcggatgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 29 - protein, 280 aa, artificial
  Synthesized ANPxy-L4-Fc2ab (protein) + signal sequence
METDTLLLWVLLLWVPGSTGSLRRSSCFGGRMDRIGAQSGLGCNSFRYGGSGGSGGSGGCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - DNA, 867 bp, artificial
  Synthesized ANPxy-L6-Fc2ab (nucleotide) + signal sequence
atggagacagacacactcctgctatgggtactgctgctctgggttccaggttccactggtagcctgcggagatccagctgcttcgggggcaggatggacaggattggagcccagagcggactgggctgtaacagcttccggtacggcggatccggcgggtccggcggctccggcggctccggcgggtccggcggatccggcggatgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 31 - protein, 289 aa, artificial
  Synthesized ANPxy-L6-Fc2ab (protein) + signal sequence
METDTLLLWVLLLWVPGSTGSLRRSSCFGGRMDRIGAQSGLGCNSFRYGGSGGSGGSGGSGGSGGSGGCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 15 aa, artificial
  Synthesized mouse IgG kappa light chain signal sequence
METDTLLLWVPGSTG

SEQ ID NO: 33 - DNA, 783 bp, Artificial
  Synthesized ANPxy-L4-Fc1ab (nucleotide)
agcctgcggagatccagctgcttcgggggcaggatggacaggattggagcccagagcggactgggctgtaacagcttccggtacggcggatccggcgggtccggcggatccggcggatgcccaccgtgcccagctccggaactgctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - DNA, 810 bp, artificial
  Synthesized ANPxy-L6-Fc1ab (nucleotide)
agcctgcggagatccagctgcttcgggggcaggatggacaggattggagcccagagcggactgggctgtaacagcttccggtacggcggatccggcgggtccggcggctccggcggctccggcgggtccggcggatccggcggatgcccaccgtgcccagctccggaactgctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 35 - DNA, 780 bp, artificial
  Synthesized ANPxy-L4-Fc2ab (nucleotide)
agcctgcggagatccagctgcttcgggggcaggatggacaggattggagcccagagcggactgggctgtaacagcttccggtacggcggatccggcgggtccggcggatccggcggatgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 36 - DNA, 807 bp, artificial
  Synthesized ANPxy-L6-Fc2ab (nucleotide)
agcctgcggagatccagctgcttcgggggcaggatggacaggattggagcccagagcggactgggctgtaacagcttccggtacggcggatccggcgggtccggcggctccggcggctccggcgggtccggcggatccggcggatgcccaccttgcccagcaccacctgtggcaggaccttcagtcttcctcttccccccaaaacccaaggacaccctgatgatctccagaacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

(19) United States
(12) Patent Application Publication
(10) Pub. No.: US 2010/0310561 A1
(43) Pub. Date: Dec. 9, 2010

(54) NATRIURETIC FUSION PROTEINS

(75) Inventors: Keith Canada, Ridgefield, CT (US); Uwe Schoenbeck, Ridgefield, CT (US); Eugene R. Hickey, Ridgefield, CT (US); Adam Mezo, Waltham, MA (US); Kevin McDonnell, Waltham, MA (US)

Correspondence Address: K&L Gates LLP, P.O. Box 1135, CHICAGO, IL, 60690, US

(73) Assignees: BOEHRINGER INGELHEIM INTERNATIONAL GMBH, Ingelheim (DE); SYNTONIX PHARMACEUTICALS, INC., Waltham, MA (US)

(21) Appl. No.: 12/522,114

(22) PCT Filed: Jun. 3, 2008
(86) PCT No.: PCT/US08/65659

§ 371 Date: Aug. 19, 2010

Related U.S. Application Data
(60) Provisional application No. 60/942,455, filed on Jun. 6, 2007.

(51) Int. Cl. A61K 39/395; C07K 16/00; C07K 14/00; C12N 15/12; C12N 15/63; C12N 5/00; C12P 21/00; A61P 9/00; A61P 31/04; A61P 11/00; A61P 13/12

(52) U.S. Cl. 424/134.1; 530/387.3; 530/350; 536/23.4; 530/391.1; 435/320.1; 435/325; 435/69.7; 424/178.1

(57) ABSTRACT

Natriuretic peptide fusion proteins comprising natriuretic peptides linked to antibody Fc domains, nucleic acid molecules encoding the fusion proteins disclosed herein, expression vectors expressing said fusion proteins, pharmaceutical compositions comprising said fusion proteins, and methods for their therapeutic use are disclosed.

### STATEMENT REGARDING JOINT RESEARCH AGREEMENT

One or more inventions contained in this application were developed under a joint research agreement as defined in the Cooperative Research and Technology Enhancement Act of 2004 between Boehringer Ingelheim International, GmbH and Syntonix Pharmaceuticals, Inc.

### FIELD OF THE INVENTION

This invention relates to fusion proteins having diuretic, natriuretic, and vasodilatory activity, specifically natriuretic peptides linked to antibody Fc domains. The fusion proteins of this invention exhibit extended plasma half-life compared to wild type natriuretic peptides, which serve as ligands to membrane guanylyl cyclases. The invention is also directed to nucleic acid molecules encoding the fusion proteins disclosed herein, expression vectors expressing said proteins, pharmaceutical compositions comprising the fusion proteins and/or nucleic acid molecules of the present invention. Compositions according to this invention may be administered by injection. The invention also relates to methods of treating or ameliorating pathological conditions in which activation of the NPRA receptor by binding of the fusion proteins of the present invention confers a therapeutic benefit on the patient.

### BACKGROUND OF THE INVENTION

Natriuretic peptides are involved in numerous biological functions including the regulation of intravascular volume, blood pressure, natriuresis, diuresis and long bone growth. These peptides include, for example, atrial natriuretic peptide (ANP) and B-type natriuretic peptide (BNP), both of which are believed to exert their cellular effects upon binding to a cell surface glycoprotein receptor, natriuretic peptide receptor A (NPRA), which is a membrane guanylyl cyclase that catalyzes the synthesis of cGMP upon ligand binding. Among their biological functions, ANP and BNP are believed to have an effect on the cardiovascular system and may be particularly effective therapeutics for the treatment of various heart conditions including acute heart failure as well as chronic congestive heart failure.

Unfortunately, despite this promising clinical application, the therapeutic usefulness of ANP and BNP is sorely limited as endopeptidase degradation, as well as natriuretic peptide clearance receptor (NPR-C) mediated internalization, causes these proteins to have a fairly short half-life in vivo. For example, the plasma half life of ANP in humans is approximately two minutes and that of BNP is estimated to be approximately 20 minutes (Potter et al., Endocrine Reviews (2006) 27(1):42-72). Thus, previous therapeutic administration of these peptides has been limited to time consuming intravenous infusion, typically in a hospital or other medical care facility. As such, there is a need for alternative therapies, which would allow medical science to more fully realize the clinical benefits of natriuretic peptides such as ANP and BNP.

### SUMMARY OF THE INVENTION

This invention relates to novel, biologically active fusion proteins comprised of one or more natriuretic peptides linked to an Fc region of IgG or other antibody.

Also provided herein are nucleic acid molecules that encode natriuretic fusion proteins of the present invention, and expression vectors that comprise polynucleotide sequences encoding natriuretic fusion proteins, for uses that include treatment or amelioration of pathological conditions in which activation of the NPRA receptor confers a therapeutic benefit on the patient, including but not limited to diseases associated with abnormal diuretic, natriuretic and vasodilatory activity. Fusion proteins or nucleic acid molecules according to the invention may be present in compositions that include pharmaceutically acceptable excipients, carriers or diluents.

In one aspect, the present invention is directed to fusion proteins that comprise one or more natriuretic peptides bound to an Fc domain by a glycine succinate linker. As contemplated herein, when a glycine succinate linker is used to link a natriuretic peptide and a Fc domain, the glycine residue of the linker is linked to the N-terminus of the Fc domain and the succinate moeity is linked to the C-terminus of the natriuretic peptide. and/or an amino acid linker of various length and sequence. In relation to the linker, the length and composition are necessary to achieve prolonged efficacy of the natriuretic peptide. As contemplated herein, the peptide may be linked to the Fc domain in different orientations. In one orientation, the C-terminus of peptide is linked to the N-terminus of the Fc domain and in another orientation, the N-terminus of the peptide is linked to the N-terminus of the Fc domain. The Fc domain exists as a homodimer of the hinge, CH2 and CH3 regions of an IgG molecule, with the Fc domain beginning at the first N-terminal cysteine residue within the IgG hinge region and the homodimer is held together by two disulfide bonds in the hinge from the cysteine residues of CysProProCysPro (SEQ ID NO: 1).

In a further aspect, the invention comprises pharmaceutical compositions comprising pharmaceutically acceptable excipients, carriers or diluents and any of the fusion peptides described herein.

In an additional aspect, the invention is also directed to nucleic acid molecules encoding the fusion proteins disclosed herein and expression vectors expressing said proteins.

In another aspect, the invention relates to methods to treat or ameliorate pathological conditions in which activation of the NPRA receptor confers a therapeutic benefit on the patient, including, but not limited to, diseases associated with abnormal diruretic, natriuretic and vasodilatory activity and/or in which it is desirable to induce naturesis, diuresis, vasodilation or to modulate the renin-angiotensin II and aldosterone systems. These conditions include those that may be characterized by an excess in extraceullar fluid, including, but not limited to, pulmonary edema. In a particularly preferred embodiment, the invention includes methods to treat or ameliorate pathological conditions of the cardiovascular system including, but not limited to, chronic heart failure (non-ischemic), post-MI heart failure (ischemic CHF), acute MI, reperfusion injury, left ventricular dysfunction (LVD), cardiac fibrosis, diastolic heart failure, and hypertrophic cardiomyopathy. In addition, hypertensive disorders including, but not limited to hypertension, e.g., pulmonary hypertension, systolic hypertension, resistant hypertension and other cardiovascular related diseases such as diabetic nephropathy may be treated or ameliorated by the methods of the present invention. It is also contemplated herein that the fusion proteins and pharmaceutical compositions of the present invention may provide therapeutic benefit for patients undergoing coronary artery bypass graft surgery (CABG).

It is also contemplated herein that the invention includes the use of the fusion proteins of the present invention in the manufacture of a medicament for the treatment or amelioration of any of the pathological conditions provided above.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE FIGURES

FIG. 1A-B: DNA and protein sequences of 4 recombinantly-produced ANP-Fc fusion proteins. The bolded mouse IgG kappa light chain signal sequence is cleaved off and not part of the final protein product. hANP28 is underlined. The (GGS)ₓGG linker is italicized.

FIG. 2: Representative dose response curve for cGMP assay. Recombinantly-produced fusion proteins are assayed for cGMP production in rat NPRA expressing 293T cells.

FIG. 3: ANP or Fusion Protein Induced cGMP Production in hNPRA 293 Cells+/−NEP.

### DETAILED DESCRIPTION OF THE INVENTION

The present disclosure provides fusion proteins that comprise a natriuretic peptide and an antibody Fc domain, wherein said natriuretic peptide is conjugated to the Fc domain directly or through a linker. The natriuretic peptide and Fc region of the fusion proteins serve two distinct biological roles that contribute to efficacy of the fusion proteins. Surprisingly, linker length also influences efficacy of the fusion proteins.

The present disclosure also provides fusion proteins that comprise at least two natriuretic peptides separated from each other by an antibody Fc domain, wherein said natriuretic peptides are conjugated to the Fc domain directly or through a linker.

In some embodiments, the fusion protein comprises the following formula:

X-Lₐ-F:F-Lₐ-X or X-Lₐ-F:F,
wherein,
X is a natriuretic peptide;
L is a linker comprising “a” amino acid residues;
a is an integer of at least 0;
: is a chemical association or crosslink; and
F is at least a portion of an immunoglobulin Fc domain comprising an FcRn binding site.

In some embodiments, the fusion protein comprises the following formula:

X-Lₐ-F:F-La-X,
wherein
X is one or more natriuretic peptides;
L is a linker comprising amino acid residues;
a is an integer of at least 0;
: is a chemical association or crosslink; and
F is at least a portion of an immunoglobulin Fc domain comprising an FcRn binding site.

In some embodiments, the natriuretic peptide is selected from the group consisting of ANP, BNP, Urodilatin, DNP or a biologically active sequence variant thereof. In some embodiments, the natriuretic peptide is ANP or BNP.

In some embodiments, the fusion protein comprises at least two natriuretic peptides. In some embodiments, x may be more than one natriuretic peptide. In some embodiments, both natriuretic peptides are ANP. In some embodiments, both natriuretic peptides are BNP.

In certain embodiments, the chemical association, i.e., (:) is a covalent bond. In other embodiments, the chemical association, i.e., (:) is a non-covalent interaction, e.g., an ionic interaction, a hydrophobic interaction, a hydrophilic interaction, a Van der Waals interaction or a hydrogen bond.

In some embodiments, the fusion protein comprises at least two Fc domains.

In some embodiments, the linker is at least 2, 4, 6, 9, 11, 16 or 20 amino acids in length. In other embodiments, the linker is at least 0, 1, 5, 7, 8, 10, 12, 13, 14, 15, 17, 18, 19, 21, 22, 23, 24, 25, 26, 27, 28, 29 or 30 amino acids, but may optionally be longer, e.g., between 30 and 40 amino acids in length or between 40 and 50 amino acids in length. In some embodiments, the linker is selected from 6 to 11 amino acids in length, 11 to 16 amino acids in length, 9 to 20 amino acids in length, 16 to 20 amino acids in length, 16 to 25 amino acids in length, 20 to 30 amino acids in length, 25 to 35 amino acids in length, 30 to 50 amino acids in length, 30 to 40 amino acids in length or 35 to 45 amino acids in length. In some embodiments, the linker is more than 10, more than 15, more than 20, more than 25, or more than 30 amino acids in length. In some embodiments, the linker is selected from a glycine succinate linker (L1), an amino acid linker or combination thereof. In some embodiments, the amino acid linker is GlyGly (L2), Gly(SerGlyGly)₂SerGly (L3) (SEQ ID NO. 2), (GlyGlySer)₃GlyGly (L4) (SEQ ID NO. 3), (GlyGlySer)₄GlyGly (SEQ ID NO. 4), (GlySerGly)₅Gly (L5a) (SEQ ID NO: 5), (GlyGlySer)₅Gly (L5) (SEQ ID NO. 6) or (GlyGlySer)₆GlyGly (L6) (SEQ ID NO. 7).

In some embodiments, the fusion protein is more resistant to proteolytic degradation than a corresponding wild type natriuretic protein. In some embodiments, the fusion protein displays a longer half-life than a corresponding wild type natriuretic protein. In some embodiments, the fusion protein is made by recombinant techniques, synthetic chemistry or semi-synthetic chemistry.

The present disclosure provides natriuretic fusion proteins that comprise any one of SEQ ID NOS. 8-11 (coded by SEQ ID NOS: 33-36, respectively). The present disclosure also provides natriuretic fusion proteins that comprise any one of SEQ ID NOS. 12-13.

The present disclosure also provides isolated polypeptides that exhibit at least 90% sequence identity with a polypeptide having a sequence selected from the group consisting of SEQ ID NO. 8, SEQ ID NO. 9, SEQ ID NO. 10 and SEQ ID NO. 11.

The present disclosure also provides isolated polypeptides that exhibit at least 90% sequence identity with a polypeptide having a sequence selected from the group consisting of SEQ ID NO. 12 and SEQ ID NO. 13.

The present disclosure also provides isolated polypeptides that exhibit at least 95% sequence identity with a polypeptide having a sequence selected from the group consisting of SEQ ID NO. 8, SEQ ID NO. 9, SEQ ID NO. 10 and SEQ ID NO. 11.

The present disclosure also provides isolated polypeptides that exhibit at least 95% sequence identity with a polypeptide having a sequence selected from the group consisting of SEQ ID NO. 12 and SEQ ID NO. 13.

The present disclosure also provides isolated polypeptides that exhibit at least 99% sequence identity with a polypeptide having a sequence selected from the group consisting of SEQ ID NO. 8, SEQ ID NO. 9, SEQ ID NO. 10 and SEQ ID NO. 11.

The present disclosure also provides isolated polypeptides that exhibit at least 99% sequence identity with a polypeptide having a sequence selected from the group consisting of SEQ ID NO. 12 and SEQ ID NO. 13.

The present disclosure provides pharmaceutical compositions comprising a natriuretic fusion protein as described herein. In some embodiments, the fusion protein is adapted for intravenous, subcutaneous or oral administration.

The present disclosure provides isolated nucleic acid molecules that encode a polypeptide selected from the group consisting of SEQ ID NO. 8, SEQ ID NO. 9, SEQ ID NO. 10 and SEQ ID NO. 11. The present disclosure also provides isolated nucleic acid molecules that encode a polypeptide selected from the group consisting of SEQ ID NO. 12 and SEQ ID NO. 13. In some embodiments, the fusion protein is recombinantly produced by employing mammalian, prokaryotic, yeast, plant, or transgenic expression systems.

The present disclosure provides methods for treating or ameliorating a condition characterized by an excessive level of extracellular fluid by administering to a subject in need thereof a therapeutically effective amount of the pharmaceutical composition that comprises one or more of the natriuretic fusion peptides as described herein.

The present disclosure provides methods for treating or ameliorating a pathological condition in which activation of the NPRA receptor confers a therapeutic benefit by administering to a subject in need thereof a therapeutically effective amount of the pharmaceutical composition that comprises one or more of the natriuretic fusion peptides as described herein.

The present disclosure provides methods for treating or ameliorating a disease associated with abnormal diruretic, natriuretic and vasodilatory activity by administering to a subject in need thereof a therapeutically effective amount of the pharmaceutical composition that comprises one or more of the natriuretic fusion peptides as described herein.

The present disclosure provides methods for treating or ameliorating a disease in which it is desirable to induce naturesis, diuresis, vasodilation or to modulate the renin-angiotensin II and aldosterone systems by administering to a subject in need thereof a therapeutically effective amount of the pharmaceutical composition that comprises one or more of the natriuretic fusion peptides as described herein.

The present disclosure provides methods for treating or ameliorating a pathological condition of the cardiovascular system selected from the group consisting of chronic heart failure (non-ischemic), reperfusion injury, left ventricular dysfunction (LVD), cardiac fibrosis, diastolic heart failure, and hypertrophic cardiomyopathy by administering to a subject in need thereof a therapeutically effective amount of the pharmaceutical composition that comprises one or more of the natriuretic fusion peptides as described herein.

The present disclosure provides methods for treating or ameliorating a hypertensive disorder selected from the group consisting of hypertension, pulmonary hypertension, systolic hypertension and resistant hypertension by administering to a subject in need thereof a therapeutically effective amount of the pharmaceutical composition that comprises one or more of the natriuretic fusion peptides as described herein.

The present disclosure provides methods for treating or ameliorating diabetic nephropathy by administering to a subject in need thereof a therapeutically effective amount of the pharmaceutical composition that comprises one or more of the natriuretic fusion peptides as described herein.

### A. Definitions

Unless otherwise defined, all technical and scientific terms used herein have the same meaning as commonly understood by one of ordinary skill in the art to which the invention belongs. The terminology used in the description of the invention herein is for the purpose of describing particular embodiments only and is not intended to be limiting of the invention. Headers are used for the convenience of the reader and are also not intended to be limiting of the invention. All publications, patent applications, patents, and other references mentioned herein are incorporated herein by reference in their entirety, as are the package inserts of any branded drugs referred to herein by their brand names.

As used in the specification and the embodiments set forth herein, the following terms have the meanings indicated.

Singular forms “a”, “an”, and “the” are intended to include the plural forms as well, unless the context clearly indicates otherwise.

A “fusion protein”, e.g. a “natriuretic fusion protein” as the term is used herein, refers to a protein having at least two polypeptides covalently linked, in which one polypeptide comes from one protein sequence or domain and the other polypeptide comes from another protein sequence or domain. Generally, the polypeptides of a fusion protein may be linked either directly or via a covalent linker The term (“linker”) refers to an amino acid linker, such as a polyglycine linker, or another type of chemical linker, e.g., a glycine succinate linker, a carbohydrate linker, a lipid linker, a fatty acid linker, a polyether linker, etc. The linker may consist of at least 2, 4, 6, 9, 11, 16 or 20 amino acids in length. Alternatively, the linker may consist of at least 0, 1, 5, 7, 8, 10, 12, 13, 14, 15, 17, 18, 19, 21, 22, 23, 24, 25, 26, 27, 28, 29 or 30 amino acids, but may optionally be longer, e.g., between 30 and 40 amino acids in length or between 40 and 50 amino acids in length. Amino acids are selected from the 20 naturally occurring amino acids, of either isomeric form D or L, for example, glycine, alanine, proline, asparagine, glutamine, and lysine. A linker may be made up of a majority of amino acids that are sterically unhindered, such as glycine and alanine The linker may comprise a range of lengths amino acid residues, such as for example, 6 to 11 amino acids in length, 11 to 16 amino acids in length, 16 to 20 amino acids in length, 16 to 25 amino acids in length, 20 to 30 amino acids in length, 30 to 35 amino acids in length, 35 to 40 amino acids in length, 40 to 45 amino acids in length or 45 to 50 amino acids in length. Some of these amino acids may be glycosylated. Non-peptide linkers are also possible. For example, alkyl linkers such as —NH—(CH₂)ₛ—C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker, wherein the linker has a molecular weight of 100 to 5000 kD, preferably 100 to 500 kD. The peptide linkers may be altered to form derivatives in the same manner as described above. As described in the examples herein, the preferred linker of the fusion proteins of the present invention is a stretch of amino acids with the basic repeat (GGS)ₓ or (GGS)ₓGG. For example, x may be an integer from 0 to 16. Although specific orientations are described in detail herein, polypeptides forming fusion proteins may be linked C-terminus to N-terminus, although they can also be linked C-terminus to C-terminus, N-terminus to N-terminus, or N-terminus to C-terminus and the polypeptides of the fusion protein can be in any order. It is also contemplated herein that the fusion proteins of the present invention may contain two peptide fusions. For example, the fusion protein may comprise one peptide flanked by two Fc domains, e.g., Fc-Natriuretic Peptide-Fc, where the natriuretic peptide is conjugated to the Fc domains directly or through a linker. As contemplated herein, when a glycine succinate linker is used to link a natriuretic peptide and a Fc domain, the glycine residue of the linker is linked to the N-terminus of the Fc domain and the succinate moeity is linked to the C-terminus of the natriuretic peptide.

The term “fusion protein” also refers to conservatively modified variants, polymorphic variants, alleles, mutant, subsequences and interspecies homologues of the polypeptides that make up the fusion protein. Fusion proteins may be produced by covalently linking a chain of amino acids from one protein sequence to a chain of amino acids from another protein sequence, e.g., by preparing a recombinant polynucleotide contiguously encoding the fusion protein or by synthetic means familiar to one of skill in the art.

The term “protein” is used herein interchangeably with “polypeptide” and “peptide.”

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Such analogs are familiar to one of skill in the art and include, e.g, phosphorothioates and phosphoramidates. Unless otherwise indicated, a particular nucleic acid sequence also implicitly encompasses conservatively modified variants thereof (e.g., degenerate codon substitutions) and complementary sequences, as well as the sequence explicitly indicated. As used herein, the term “nucleic acid” may also be referred to as “gene”, “cDNA”, “mRNA”, “oligonucleotide”, and “polynucleotide”.

As contemplated herein, a polynucleotide sequence comprising a fusion protein of the present invention hybridizes under stringent conditions to each of the nucleotide sequences encoding each individual polypeptide of the fusion protein. The polynucleotide sequences encoding the individual polypeptides of the fusion polypeptide therefore include conservatively modified variants, polymorphic variants, alleles, mutants, subsequences, and interspecies homologs.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99% or higher, compared to a reference sequence using the programs familiar to one of skill in the art; preferably BLAST using standard parameters. One of skill in the art will recognize that these values can be appropriately adjusted to determine corresponding identity of proteins encoded by two nucleotide sequences by taking into account codon degeneracy, amino acid similarity, reading frame positioning and the like. “Substantial identity” of amino acid sequences for these purposes normally means sequence identity of at least 40%. Preferred percent identity of polypeptides can be any integer from 40% to 100%. More preferred embodiments include at least 60%, 65%, 70%, 75%, 80%, 85%, 90%, 95%, or 99%. Polypeptides that are “substantially similar” share sequences as noted above except that residue positions that are not identical may differ by conservative amino acid changes.

Optimal alignment of sequences for comparison may be conducted according to conventional methods, e.g., by the local identity algorithm of Smith and Waterman (1981) Add. APL. Math. 2:482, by the identity alignment algorithm of Needleman and Wunsch (1970) J. Mol. Biol. 48:443, by the search for similarity method of Pearson and Lipman (1988) Proc. Natl. Acad. Sci. (U.S.A.) 85: 2444, or by computerized implementations of these algorithms (GAP, BESTFIT, BLAST, FASTA, and TFASTA in the Wisconsin Genetics Software Package, Genetics Computer Group (GCG), 575 Science Dr., Madison, Wis.).

A preferred example of algorithms that are suitable for determining percent sequence identity and sequence similarity are the BLAST and BLAST 2.0 algorithms, which are described in Altschul et al. (1977) Nuc. Acids Res. 25:3389 3402 and Altschul et al. (1990) J. Mol. Biol. 215:403 410, respectively and are familiar to one of skill in the art.

Another indication that nucleotide sequences are substantially identical is if two molecules hybridize to each other, or to a third nucleic acid, under moderately, and preferably highly, stringent conditions. Generally, hybridization conditions of high, moderate and low stringency are familiar to one of skill in the art. For example, stringent conditions are selected to be about 5 to 10° C. below the temperature under defined ionic strength and pH at which 50% of the target sequence hybridizes to a perfectly matched probe (Tₘ); medium stringency is about 20-29° C. below the Tₘ and low stringency conditions are characterized by temperature that is about 40-48° C. below the Tₘ. An example of stringent hybridization conditions may be considered to be 50% formamide, 5×SSC, and 1% SDS, incubating at 42° C., or 5×SSC, 1% SDS, incubating at 65° C. and washing in 0.2×SSC, and 0.1% SDS at 65°. See, e.g, Tijssen, Techniques in Biochemistry and Molecular Biology—Hybridization with Nucleic Probes, “Overview of principles of hybridization and the strategy of nucleic acid assays” (1993); Guide to Molecular Cloning Techniques, Methods in Enzymology, Vol 152, 1987, Berger and Kimmel, eds.).

“Amino acid” is defined herein as any naturally occurring, artificial, or synthetic amino acid in either its L or D stereoisomeric forms, unless otherwise specified. The term “residue” is used interchangeably with the term “amino acid,” and is often designated as having a particular position in a given sequence of amino acids.

“Biologically active” refers to an agent having therapeutic or pharmacologic activity, such as an agonist, partial agonist or antagonist.

“Effective amount” as provided herein refers to a nontoxic but sufficient amount to provide the desired therapeutic effect. As will be pointed out below, the exact amount required will vary from subject to subject, depending on age, general condition of the subject, the severity of the condition being treated, the particular biologically active agent administered, and the like. An appropriate “effective” amount in any individual case may be determined by one of ordinary skill in the art by reference to the pertinent texts and literature and/or by using routine experimentation.

As used herein, the term “Fc domain” refers to that part of an antibody derived from the stem of the “Y,” and is composed of two heavy chains that each contribute two to three constant domains (depending on the class of the antibody). The Fc region binds to various cell receptors and complement proteins, and mediates different physiological effects of antibodies. As contemplated herein, the Fc domain of any antibody which displays minimal to no effector function may be used with the present invention. These include, but are not limited to, IgG1, IgG2, IgG4 but may also include any Fc domain of any antibody, the sequence of which has been altered, according to methods familiar to one of skill in the art, to possess minimal effector activity.

Alternatively, the term “Fc domain” may be described as an IgG heavy chain comprising hinge, CH2 and CH3 regions, wherein the IgG heavy chain begins at the first N-terminal cysteine residue within the hinge region and forms a homodimer with another Fc domain at the first and second N-terminal cysteine residues.

The term “Fc” is also used to describe part of the fusion proteins. In this context, Fc is an IgG heavy chain comprising hinge, CH2 and CH3 domains, the IgG heavy chain begins at the first N-terminal cysteine residue within the hinge region and forms a homodimer with another Fc at the first and second N-terminal cysteine residues. In other words, the N-terminal amino acid sequence of each chain of the Fc homodimer begins with CysProProCysPro (SEQ ID NO: 1) of the IgG hinge region and both Cys residues are disulfide bonded.

As used herein, a peptide is said to be “isolated” or “purified” when it is substantially free of cellular material or free of chemical precursors or other chemicals. The fusion peptides of the present invention can be purified to homogeneity or other degrees of purity. The level of purification will be based on the intended use. The critical feature is that the preparation allows for the desired function of the peptide, even if in the presence of considerable amounts of other components.

“Natriuretic peptides” as referred to herein include the mammalian natriuretic factors (ANP, BNP, CNP), as well as Salmon cardiac peptide (Tervonen et al., Endocrinology 139(9):4021-4025 (1998)) and Dendroaspis natriuretic peptide (DNP) (Schweitz et al. J. Biol. Chem., 267(20):13928-13932 (1992)), urodilatins and peptides analogous thereto, and analogs, active fragments, degradation products, salts, variants, derivatives and combinations thereof. Specifically, human ANP and BNP include “hANP28” and “hBNP32” as disclosed in Kangawa et al., Biochem. Biophys. Res. Comm., 118(1):131-139 (1984), Sudoh, et al. Nature 332(6159):78-81 (1988), and Kambayashi et al. FEBS Lett., January 1; 259(2):341-5 (1990).

“Natriuretic peptides” include peptides that exhibit natriuretic activity, including, for example, peptides that are allelic variants, orthologs, splice variants, and/or species homologues of a natriuretic peptide. Procedures known in the art can be used to obtain full-length genes and cDNAs, allelic variants, splice variants, full-length coding portions, orthologs, and/or species homologues of genes and cDNAs corresponding to a nucleotide sequence coding for a natriuretic peptide. For example, allelic variants, orthologs and/or species homologues may be isolated and identified by making suitable probes or primers from the sequences provided herein and screening a suitable nucleic acid source for allelic variants and/or the desired homologue using any technique known to those skilled in the art.

Without being limited by any particular mode of action, as referred to herein, the pharmacokinetics of a “sustained-release, or depot formulation” may be characterized as displaying an increase in bioavailability, due to FcRn binding and recycling of FcRn-bound molecules from within acidic lysosomes back to the general circulation (V. Ghetie and E. S. Ward, Annual Rev. Immunol., 18, 739-766, (2000)).

The term “semi-synthetic” as used herein refers to a process to synthesize the fusion proteins of the present invention comprising the use of both synthetic chemistry and recombinant techniques. For example, the Fc domain of the fusion proteins disclosed herein may be made recombinantly, while the natriuretic peptide and linker may be made synthetically.

### B. Peptide Molecules

This invention relates to novel, biologically active fusion proteins comprised of one or more natriuretic peptides linked to an Fc region of IgG or other antibody for uses that include treatment or amelioration of pathological conditions in which activation of the NPRA receptor confers a therapeutic benefit on the patient, including but not limited to diseases associated with abnormal diruretic, natriuretic and vasodilatory activity. Fusion proteins according to the invention may be present in compositions that include pharmaceutically acceptable excipients, carriers or diluents.

The invention relates to fusion proteins as described herein that may have one of the following general formulas, A or B,

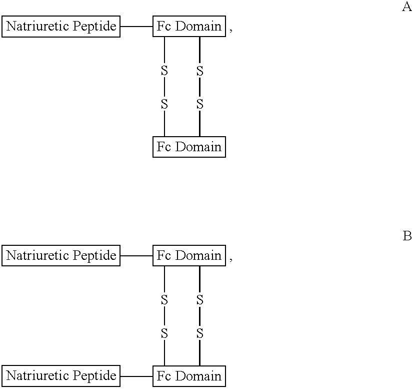

wherein,

the natriuretic peptide (i) is selected from the group consisting of one or more ANP, BNP, urodilatin, DNP and a biologically active sequence variant thereof and (ii) may be in an orientation of N′ to C′ of the amino acid sequence, C′ to N′ of the amino acid sequence or in the case of more than one natriuretic peptide, N′ to C′, C′ to N′ or a mixture of N′ to C′ and C′ to N′; and

the Fc domain is an IgG heavy chain comprising hinge, CH2 and CH3 regions, wherein the IgG heavy chain begins at the first N-terminal cysteine residue within the hinge region and forms a homodimer with another Fc domain at the first and second N-terminal cysteine residues (e.g., SEQ ID NO: 14 or SEQ ID NO: 17), and wherein the Fc domain is denoted by Fc_{AB} or Fc_{BA}, wherein AB is an orientation of N′ to C′ of both Fc domains and BA is an orientation of C′ to N′ of both Fc domains.

As contemplated herein, in one aspect this invention embodies fusion proteins comprising at least one natriuretic peptide conjugated to the Fc domain of an antibody by way of a linker. The fusion protein may actually comprise one natriuretic peptide or two natriuretic peptides conjugated to an antibody Fc domain. As described in detail below, the sequence and length of the linker employed to conjugate the peptide with the Fc domain may vary depending on whether the fusion protein comprises one or two natriuretic peptides.

One aspect of the invention relates to a fusion protein having one of the following general formulas, 1 or 2,

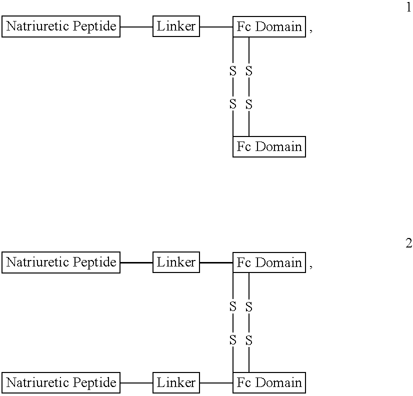

wherein,

the natriuretic peptide (i) is selected from the group consisting of one or more ANP, BNP, urodilatin, DNP and a biologically active sequence variant thereof and (ii) may be in an orientation of N′ to C′ of the amino acid sequence, C′ to N′ of the amino acid sequence or in the case of more than one natriuretic peptide, N′ to C′, C′ to N′ or a mixture of N′ to C′ and C′ to N′;

the linker is one or more linkers selected from the group consisting of a succinate-glycine linker (L1), a GlyGly linker (L2), a Gly(SerGlyGly)₂SerGly linker (L3) (SEQ ID NO. 2) a (GlyGlySer)_{y}GlyGly linker, wherein y is 3 to 6 (e.g., SEQ ID NOS. 3 (L4), 4 and 7 (L6)) (GlyGlySer)₅Gly (L5) (SEQ ID NO. 6) and a (GlySerGly)₅Gly linker (L5a) (SEQ ID NO: 5); and

the Fc domain is an IgG heavy chain comprising hinge, CH2 and CH3 regions, wherein the IgG heavy chain begins at the first N-terminal cysteine residue within the hinge region and forms a homodimer with another Fc domain at the first and second N-terminal cysteine residues (e.g., SEQ ID NO: 14 or SEQ ID NO: 17), and wherein the Fc domain is denoted by Fc_{AB} or Fc_{BA}, wherein AB is an orientation of N′ to C′ of both Fc domains and BA is an orientation of C′ to N′ of both Fc domains.

In another aspect of this invention, the fusion protein has the following formula 3,

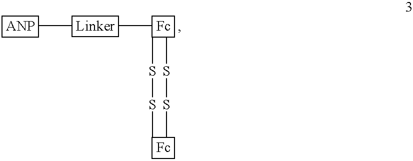

wherein,

ANP is in an orientation of N′ to C′ (ANP_{XY}) of the amino acid sequence of ANP (SEQ ID NO. 15) or in an orientation of C′ to N′ (ANP_{YX}) of the amino acid sequence of ANP;

the linker is one or more linkers selected from the group consisting of L1, L2, L3, L4, L5, L5a and L6, wherein L1 is a glycine succinate linker as described herein, L2 is a GlyGly linker, L3 is a Gly(SerGlyGly)₂SerGly linker (SEQ ID NO. 2), L4 is a (GlyGlySer)₃GlyGly linker (SEQ ID NO. 3), L5 is a (GlyGlySer)₅Gly linker (SEQ ID NO. 6), L5a is a (SerGlyGly)₅Gly (SEQ ID NO. 5) and L6 is a (GlyGlySer)₆GlyGly linker (SEQ ID NO. 7); and

Fc is (i) an IgG heavy chain comprising hinge, CH2 and CH3 regions, wherein the IgG heavy chain begins at the first N-terminal cysteine residue within the hinge region and forms a homodimer with another Fc at the first and second N-terminal cysteine residues (e.g., SEQ ID NO: 14 and SEQ ID NO: 17), and (ii) is denoted by Fc1_{AB}, Fc1_{BA}, Fc2_{AB} or Fc2_{BA}, wherein Fc1 is derived from an IgG1 molecule, Fc2 is derived from a IgG2 molecule, AB is an orientation of N′ to C′ of the Fc and BA is an orientation of C′ to N′ of the Fc.

In yet another aspect of this invention, the fusion protein has the following formula 4,

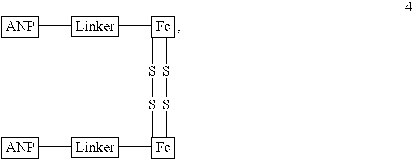

wherein,

ANP is in an orientation of N′ to C′ (ANP_{XY}) of the amino acid sequence of ANP (SEQ ID NO. 15) or in an orientation of C′ to N′ (ANP_{YX}) of the amino acid sequence of ANP;

Linker is one or more linkers selected from the group consisting of L1, L2, L3, L4, L5, L5a, and L6 wherein L1 is a glycine succinate linker as described herein, L2 is a GlyGly linker, L3 is a Gly(SerGlyGly)₂SerGly linker (SEQ ID NO. 2), L4 is a (GlyGlySer)₃GlyGly linker (SEQ ID NO. 3), L5 is a (GlyGlySer)₅Gly linker (SEQ ID NO. 6), L5a is a (SerGlyGly)₅Gly (SEQ ID NO. 5) and L6 is a (GlyGlySer)₆GlyGly linker (SEQ ID NO. 7); and

Fc is (i) an IgG heavy chain comprising hinge, CH2 and CH3 regions, wherein the IgG heavy chain begins at the first N-terminal cysteine residue within the hinge region and forms a homodimer with another Fc at the first and second N-terminal cysteine residues (e.g., SEQ ID NO: 14 or SEQ ID NO: 17), and (ii) is denoted by Fc1_{AB}, Fc1_{BA}, Fc2_{AB} or Fc2_{BA}, wherein Fc1 is derived from an IgG1 molecule, Fc2 is derived from a IgG2 molecule, AB is an orientation of N′ to C′ of the Fc and BA is an orientation of C′ to N′ of the Fc.

The fusion proteins of the present invention are biologically active molecules, e.g., they are able to catalyze cGMP, but are more useful for therapeutic purposes as they possess much longer half-lives and are also less susceptible to proteolytic degradation. In addition, by exploiting FcRn mediated transport, the therapeutic fusion proteins disclosed herein may be administered by bolus injection but may display pharmacokinetic properties resembling that of a slow-release depot formulation.

The fusion proteins of the present invention are natriuretic peptides that are conjugated to a Fc region of an antibody, such as IgG, directly or through a linker. By conjugating the peptide to the Fc region of an antibody, these fusion proteins exhibit much longer half-lives than the unconjugated peptides. Without being limited by any particular mode of action, the fusion proteins of the present invention may be pinocytosed and sequestered upon binding of the Fc region to the neonatal constant region fragment receptor (FcRn) and by exploiting the FcRn active carrier system, (the FcRn pathway transports maternal antibodies (IgG) across the intestinal epithelium of a newborn animal), levels of the fusion proteins disclosed herein can be protected from intracellular lysozomal degredation as well as have reduced exposure to neutral endopeptidase (NEP) or the NPR clearance receptor. The fusion protein may be recycled and represented to the circulation upon normal release from the cell. In this way, activation of the NPRA receptor all at once, such as typical after a bolus dose of ligand, may be avoided. The bioavailability of the fusion proteins of the present invention may more closely resemble a slow-release depot preparation.

The FcRn receptor is expressed on the surface of endothelial cells in several different types of tissue in adult humans, including lung, kidney and intestine. Without being limited by any particular mode of action, the normal function of the FcRn receptor may be exploited as a means to administer bioactive ANP-Fc and BNP-Fc fusion proteins in a human for a myriad of clinical uses. For example, in addition to methods to treat or ameliorate pathological conditions of the cardiovascular system, the fusion proteins of the present invention may be used in methods to treat diseases associated with abnormal diuretic, natriuretic and vasodilatory activity in which activation of the NPRA receptor confers a therapeutic benefit on the patient.

The fusion proteins of the present invention may comprise any natriuretic peptide, but are preferably ANP or BNP. Human versions of these proteins are known as, e.g., hANP28 (Kangawa et al. Biochem. Biophys. Res. Comm. 118(1):131-139 (1984) of SEQ ID NO. 15 (NCBI database accession number NM_{—}006172) and hBNP32 (Kambayashi et al. FEBS Lett 1990 Jan. 1; 259(2):341-5) of SEQ ID NO. 16 (NCBI database accession number NM_{—}002521). In addition to various structural and sequence forms of the wild type natriuretic peptides, it is understood that the present invention contemplates any and all possible biologically active sequence variants, whether naturally occurring or synthesized by design. It is further understood that peptides from any species are included within the scope of the present invention, although human peptides are preferred. In addition, the fusion proteins of the present invention may comprise any possible combination between human and non-human polypeptides, e.g., from animals such as, rat, mouse, guinea pig, horse, cow, pig, sheep, dog, etc.

It is also contemplated that in addition to sequence variants, fragments of said peptides are included within the scope of the invention disclosed herein, where such fragments are of sufficient size to be therapeutically effective in the methods of the present invention. One of skill in the art can determine alterations in peptide length and sequence variety that does not impact biological activity and/or therapeutic effectiveness without undue experimentation. The proteins may be in the form of acidic or basic salts, or may be in a neutral form. Individual amino acid residues may also be modified by oxidation or reduction.

Other variants within the scope of the invention include fusion proteins in which the primary amino acid structure is modified by forming covalent or aggregative conjugates with other peptides or polypeptides, or chemical moieties such as glycosyl groups, lipids, phosphate, acetyl groups and the like. Covalent derivatives may be prepared, for example, by linking particular functional groups to amino acid side chains or at the N- or C-terminus.

The fusion proteins of the present invention may or may not be glycosylated. Fusion proteins expressed in yeast or mammalian expression systems may be similar to, or slightly different in molecular weight and glycosylation pattern from the native molecules, depending upon the expression system; expression of DNA encoding polypeptides in bacteria such as E. coli provides non-glycosylated molecules.

In certain embodiments, dimer constructs of the present invention have been found to possess increased in vitro potency when compared to monomer constructs. The increased potency of dimer constructs of natriuretic peptides linked to antibody Fc domains as described herein is particularly surprising in view of the monomeric interaction between the natriuretic peptide ligands (e.g., ANP, BNP, etc.) and their cell surface receptors. It would be expected that dimeric constructs as described herein would be sterically hindered from interacting with the cells and/or receptors and thus, little and/or no activity of such dimeric constructs would be predicted.

In certain embodiments, monomer constructs of the present invention have been found to possess increased in vivo serum concentrations (Cmax) when compared to dimer constructs. The increased Cmax of monomer constructs of natriuretic peptides linked to antibody Fc domains as described herein is surprising in view of the previous results from intravenous administration of monomeric EPO-Fc constructs that showed a lower Cmax as compared to intravenous administration of dimeric EPO-Fc constructs (see, e.g., Table 4 of U.S. Patent Application Publication No. 2007/0172928).

The Fc domain conjugated to the natriuretic peptide(s) is preferably the Fc domain of IgG, including, but not limited to, IgG1, IgG2 or IgG4 (see, e.g., SEQ ID NO: 14 or SEQ ID NO: 17). However, Fc domains of other antibodies may be used if modified to possess minimal or no effector function. Human antibody Fc domains are preferred, but other species types, wild-type forms as well as sequence variants, may be used, e.g, a recombinant Fc molecule is described in the Examples provided herein. In one aspect of this invention, the Fc domain is made up of two Fc heavy chains from IgG1 or IgG2 isotypes with the hinge residues removed down to the CPPCP sequence on each chain to allow for interchain disulfide bonding of the cysteine residues. Ideally, the natriuretic fusion proteins of the present invention comprise an Fc domain that is able to bind to the FcRn receptor, trigger the active carrier function of this receptor and cause delivery of the fusion protein into the cell. Once inside the cell, pH changes result in the release of the fusion protein from the FcRn receptor, and the fusion protein may ultimately be released from the cell back into the circulation. (Roopenian D. C. et. al. (2003) J. Immunology 170:3528-3533; Lencer, W. I. et al, Trends in Cell Biology 15(1):5-9 (2005)).

As shown herein, the length and sequence of the amino acid linker used to conjugate the natriuretic peptide with the Fc domain vary. As described in the Examples provided below, structural modeling of NPRA with ANP bound in relation to an Fc domain was used to predict the minimum linker distance required to allow insertion of the Fc-fused ANP into the NPRA active site. As contemplated herein, desirable linker length is one that minimizes steric and electrostatic repulsions between the natriuretic peptide and the Fc domain. For example, the desired minimal distance from the C-terminus of ANP is 12 A from the closest N-terminus of the Fc homodimer and 17 A from the other N-terminus of the Fc homodimer. Furthermore, if the Fc homodimer has only one ANP fused, (e.g., monomer) a 4 to 6 amino acid minimum linker length would be preferred. With two ANP peptides bound to the Fc homodimer (e.g., dimer), and with only one ANP bound to NPRA receptor (i.e., in a 1:1 Fc dimer:NPRA ratio) then a minimum linker length of 9 amino acids for each linker is preferred. For both ANPs to bind to NPRA (e.g., to two adjacent receptors or to 1 NPRA receptor in nonstatic, alternating fashion) then linkers with a minimum length of 12 amino acids would be preferred. Surprisingly, it has been discovered that increasing linker length enabled the fusion proteins to approach the potency of the fused natriuretic peptide. For example, it was found that longer linker lengths increased potency of the natriuretic peptide (as measured by ability to induce cGMP in vitro), particularly those fusion proteins having a linker length of 20 amino acids.

Unexpectedly, it has been discovered that increasing linker length enabled the fusion proteins to approach the potency of the natriuretic peptide. Those fusion proteins having a linker length longer than predicted showed the most improved potency, particularly those fusion proteins having a linker length of, e.g., 20 amino acids (see, e.g., Examples 3 and 6). In fact, the potency of the fusion proteins with linker lengths longer than predicted by modeling approached the potency of native ANP. In embodiments of this invention, the potency of the fusion protein is improved at a linker length of between 16 and 50 amino acids, preferably between 16 and 40 amino acids and most preferably between 16 and 30 amino acids. Additionally, it would have been expected that increasing linker length would increase the fusion protein's susceptibility to proteolytic degradation by NEP. In contrast, experiments unexpectedly demonstrate that longer linker lengths are not affected by NEP (see, e.g., Example 5).

Linker sequences employed in the present invention comprising (GGS)x repeats (e.g., where x is an integer from 0 to 16), may be made according to conventional synthetic, semi-synthetic, or recombinant methods (see, e.g., Evers T. H. et. al. (2006) Biochemistry, 45:13183-13192). With regard to the actual amino acid sequence of the linkers employed, typically glycines and serines are preferred, as the presence of glycines in the linker provide flexibility and serines provide solubility. A preferred linker sequence is made up of a series of repeats of these amino acids, e.g., (GGS)x-GG, for example, where x is an integer from 0 to 16, such as GGSGGSGGSGG (SEQ ID NO. 3) or GGSGGSGGSGGSGGSGGSGG (SEQ ID NO. 7).

As mentioned above, the orientation of conjugation of Fc domain and natriuretic peptide may vary. For example, the carboxy terminus of the peptide may be linked to the amino terminus of the Fc domain by a normal peptide bond (see Table 2, may also be referred to as Orientation #1). Alternatively, the amino terminus of the peptide may be linked to the amino terminus of the Fc domain (see Table 2, may also be referred to as Orientation #2). In the latter case, the chemistry leaves a succinate moiety in place of one amino acid of the fusion. As such, one of skill in the art will recognize that fusion proteins of the latter case may not be made recombinantly as normal peptide bonding does not take place between two amino termini. Data gathered indicate that orientation does not seem to effect the potency of a given fusion protein (see, e.g., Example 2).

ANP-Fc fusion constructs of the present invention may comprise one or more ANP, one or more linkers and one or more Fc. The following ANP-Fc fusion constructs are contemplated by the present invention.

An exemplary fusion construct (Construct 2), which comprises ANP_{XY} (Construct 1) (SEQ ID NO: 15), is represented by:

ANP_{XY}-L4-Fc1_{AB}

The construct comprises ANP_{XY}-L4-Fc1_{AB}, wherein ANP_{XY} is SEQ ID NO: 15, L4 is SEQ ID NO: 3 and Fc1_{AB} is SEQ ID NO: 14. ANP_{XY}-L4-Fc1_{AB} is represented by SEQ ID NO: 8. When the ANP_{XY}-L4-Fc1_{AB} construct is made recombinantly, a homodimer may be produced, for example, the ANP_{XY}-L4-Fc1_{AB} may be linked to a second ANP_{XY}-L4-Fc1_{AB} construct via a disulfide linkage.

An exemplary fusion construct (Construct 3) is represented by:

ANP_{XY}-L6-Fc1_{AB}

The construct comprises ANP_{XY}-L6-Fc1_{AB}, wherein ANP_{XY} is SEQ ID NO: 15, L6 is SEQ ID NO: 7 and Fc1_{AB} is SEQ ID NO: 14. ANP_{XY}-L6-Fc1_{AB} is represented by SEQ ID NO: 9. When the ANP_{XY}-L6-Fc1_{AB} construct is made recombinantly, a homodimer may be produced, for example, the ANP_{XY}-L6-Fc1_{AB} may be linked to a second ANP_{XY}-L6-Fc1_{AB} construct via a disulfide linkage.

An exemplary fusion construct (Construct 4) is represented by:

ANP_{XY}-L4-Fc2_{AB}

The construct comprises ANP_{XY}-L4-Fc2_{AB}, wherein ANP_{XY} is SEQ ID NO: 15, L4 is SEQ ID NO: 3 and Fc2_{AB} is SEQ ID NO: 17. ANP_{XY}-L4-Fc2_{AB} is represented by SEQ ID NO: 10. When the ANP_{XY}-L4-Fc2_{AB} construct is made recombinantly, a homodimer may be produced, for example, the ANP_{XY}-L4-Fc2_{AB} may be linked to a second ANP_{XY}-L4-Fc2_{AB} construct via a disulfide linkage.

An exemplary fusion construct (Construct 5) is represented by:

ANP_{XY}-L6-Fc2_{AB}

The construct comprises ANP_{XY}-L6-Fc2_{AB}, wherein ANP_{XY} is SEQ ID NO: 15, L6 is SEQ ID NO: 7 and Fc2_{AB} is SEQ ID NO: 17. ANP_{XY}-L6-Fc2_{AB} is represented by SEQ ID NO: 11. When the ANP_{XY}-L6-Fc2_{AB} construct is made recombinantly, a homodimer may be produced, for example, the ANP_{XY}-L6-Fc2_{AB} may be linked to a second ANP_{XY}-L6-Fc2_{AB} construct via a disulfide linkage.

An exemplary fusion construct (Construct 6) is represented by:

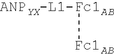

The construct comprises ANP_{YX}-L1-Fc1_{AB}, wherein ANP_{YX} is SEQ ID NO: 15 inverted in orientation from its C′ to N′ terminus, L1 is Succinate-Gly and Fc1_{AB} is SEQ ID NO: 14. The ANP_{YX}-L1-Fc1_{AB} may be linked to a second Fc1_{AB} via a disulfide linkage.

An exemplary fusion construct (Construct 7) is represented by:

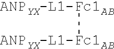

The construct comprises ANP_{YX}-L1-Fc1_{AB}, wherein ANP_{YX} is SEQ ID NO: 15 inverted in orientation from its C′ to N′ terminus, L1 is Succinate-Gly and Fc1_{AB} is SEQ ID NO: 14. The ANP_{YX}-L1-Fc1_{AB} may be linked to a second ANP_{YX}-L1-Fc1_{AB} construct via a disulfide linkage.

An exemplary fusion construct (Construct 8) is represented by:

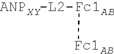

The construct comprises ANP_{XY}-L2-Fc1_{AB}, wherein ANP_{XY} is SEQ ID NO: 15, L2 is GlyGly and Fc1_{AB} is SEQ ID NO: 14. ANP_{XY}-L2-Fc1_{AB} is represented by SEQ ID NO: 13. The ANP_{XY}-L2-Fc1_{AB} may be linked to a second Fc1_{AB} via a disulfide linkage.

An exemplary fusion construct (Construct 9) is represented by:

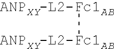

The construct comprises ANP_{XY}-L2-Fc1_{AB}, wherein ANP_{XY} is SEQ ID NO: 15, L2 is GlyGly and Fc1_{AB} is SEQ ID NO: 14. The ANP_{XY}-L2-Fc1_{AB} may be linked to a second ANP_{XY}-L2-Fc1_{AB} construct via a disulfide linkage.

An exemplary fusion construct (Construct 10) is represented by:

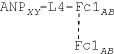

The construct comprises ANP_{XY}-L4-Fc1_{AB}, wherein ANP_{XY} is SEQ ID NO: 15, L4 is SEQ ID NO: 3 and Fc1_{AB} is SEQ ID NO: 14. The ANP_{XY}-L4-Fc1_{AB} is represented by SEQ ID NO: 13. The ANP_{XY}-L4-Fc1_{AB} may be linked to a second Fc1_{AB} via a disulfide linkage.

An exemplary fusion construct (Construct 11) is represented by:

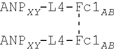

The construct comprises ANP_{XY}-L4-Fc1_{AB}, wherein ANP_{XY} is SEQ ID NO: 15, L4 is SEQ ID NO: 3 and Fc1_{AB} is SEQ ID NO: 14. The ANP_{XY}-L4-Fc1_{AB} may be linked to a second ANP_{XY}-L4-Fc1_{AB} construct via a disulfide linkage.

An exemplary fusion construct (Construct 12) is represented by:

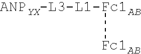

The construct comprises ANP_{YX}-L3-L1-Fc1_{AB}, wherein ANP_{YX} is SEQ ID NO: 15 inverted in orientation from its C′ to N′ terminus, L3 is SEQ ID NO: 2, L1 is Succinate-Gly and Fc1_{AB} is SEQ ID NO: 14. The ANP_{YX}-L3-L1-Fc1 may be linked to a second Fc1_{AB} via a disulfide linkage.

An exemplary fusion construct (Construct 13) is represented by:

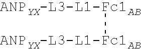

The construct comprises ANP_{YX}-L3-L1-Fc1_{AB}, wherein ANP_{YX} is SEQ ID NO: 15 inverted in orientation from its C′ to N′ terminus, L3 is SEQ ID NO: 2, L1 is Succinate-Gly and Fc1_{AB} is SEQ ID NO: 14. The ANP_{YX}-L3-L1-Fc1_{AB} may be linked to a second ANP_{YX}-L3-L1-Fc1_{AB} construct through a disulfide linkage.

An exemplary fusion construct (Construct 14) is represented by:

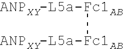

The construct comprises ANP_{XY}-L5a-Fc1_{AB}, wherein ANP_{XY} is SEQ ID NO: 15, L5a is SEQ ID NO: 5 and Fc1_{AB} is SEQ ID NO: 14. The ANP_{XY}-L5a-Fc1_{AB} may be linked to a second ANP_{XY}-L5a-Fc1_{AB} construct through a disulfide linkage.

An exemplary fusion construct (Construct 15) is represented by:

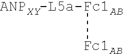

The construct comprises ANP_{XY}-L5a-Fc1_{AB}, wherein ANP_{XY} is SEQ ID NO: 15, L5a is SEQ ID NO: 5 and Fc1_{AB} is SEQ ID NO: 14. The ANP_{XY}-L5a-Fc1_{AB} may be linked to a second Fc1_{AB} via a disulfide linkage.

### C. Synthesis of the Fusion Proteins

Unless otherwise indicated herein, the fusion proteins of the present invention may be made by any of a number of techniques of protein chemistry or molecular biology familiar to one of skill in the art. (See, e.g., Dawson et al., Ann. Rev. Biochem., 69:923-960, 2000.) Possible synthesis scenarios are described in the Examples provided herein and include synthetic and semi-synthetic chemical synthesis as well as recombinant methods.

Fusion proteins may be produced using chemical methods in whole or in part and using classical or nonclassical amino acids or chemical amino acid analogs as appropriate. Techniques include solid phase chemistry (Merrifield, J. Am. Chem. Soc., 85:2149,1964; Houghten, Proc. Natl. Acal. Sci. USA 82:5132, 1985) and equipment for such automated synthesis of polypeptides is commercially available (e.g., Applied Biosystems, Foster City, Calif.). Synthesized peptides can be purified using conventional methods such as high performance liquid chromatography. The composition of the synthetic fusion polypeptides may be confirmed by amino acid analysis or sequencing using techniques familiar to one of skill in the art. Further treatment of a synthesized protein under oxidizing conditions may also be utilized to obtain the proper native conformation. See, e.g. Kelley, R. F. & Winkler, M. E. in Genetic Engineering Principles and Methods, Setlow, J. K., ed., Plenum Press, N.Y., vol. 12, pp 1-19, 1990; Stewart, J. M. & Young, J. D. Solid Phase Peptide Synthesis Pierce Chemical Co., Rockford, Ill.,1984).

The fusion proteins disclosed herein may also be made by recombinant techniques involving gene synthesis, cloning and expression methodologies. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

Briefly, the fusion proteins of the present invention may be made recombinantly by isolating or synthesizing nucleic acid sequences encoding any of the amino acid sequences described herein by conventional cloning or chemical synthesis methods. For example, DNA fragments coding for the different fusion protein sequences may be ligated together in-frame in accordance with conventional techniques or synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence. The recombinant nucleic acids can further comprise other nucleotide sequences such as sequences that encode affinity tags to facilitate protein purification protocol.

The nucleic acid sequence encoding a fusion protein of the present invention may be ligated into a suitable expression vector capable of expressing the nucleic acid sequence in a suitable host, followed by transforming the host with the expression vector into which the nucleic acid sequence has been ligated, culturing the host under conditions suitable for expression of the nucleic acid sequence, whereby the protein encoded by the selected nucleic acid sequence is expressed by the host and purifying the protein produced. In this process, the ligating step may further contemplate ligating the nucleic acid into a suitable expression vector such that the nucleic acid is operably linked to a suitable secretory signal, whereby the amino acid sequence is secreted by the host. Suitable secretory signals for use with the present invention include but are not limited to, the mouse IgG kappa light chain signal sequence (Ho et. al. PNAS (2006) 103(25): 9637-9642).

As described above, a nucleic acid sequence encoding a fusion protein described herein may be inserted into an appropriate plasmid or expression vector that may be used to transform a host cell. In general, plasmid vectors containing replication and control sequences that are derived from species compatible with the host cell are used in connection with those hosts. The vector ordinarily carries a replication site, as well as sequences which encode proteins that are capable of providing phenotypic selection in transformed cells. For example, E. coli may be transformed using pBR322, a plasmid derived from an E. coli species (Mandel, M. et al., J. Mol. Biol. 53:154,1970). Plasmid pBR322 contains genes for ampicillin and tetracycline resistance, and thus provides easy means for selection. Other vectors include different features such as different promoters, which are often important in expression. The vectors used for mammalian expression often contain the constitutive CMV promoter that leads to high recombinant protein expression. These vectors also contain selection sequence genes that are used for the generation of stable expressing cell lines.

Host cells may be prokaryotic or eukaryotic. Prokaryotes are preferred for cloning and expressing DNA sequences to produce parent polypeptides, segment substituted polypeptides, residue-substituted polypeptides and polypeptide variants. Such prokaryotic cells familiar to one skilled in the art include, but are not limited to, E. coli, B. subtillus, and P. aeruginosa cell strains. In addition to prokaryotes, eukaryotic organisms, such as yeast cultures, or cells derived from multicellular organisms may be used. Vertebrate cells may also be used as useful host cell lines. Useful cells and cell lines are familiar to one of skill in the art and include, but are not limited to, HEK293 cells, HeLa cells, Chinese Hamster Ovary (CHO) cell lines, W138, 293, BHK, COS-7 and MDCK cell lines.

The invention also relates to isolated or purified polynucleotides that encode the natriuretic fusion proteins of the present invention. As discussed above, the polynucleotides of the invention which encode a fusion protein, fragments thereof, or functional equivalents thereof may be used to generate recombinant nucleic acid molecules that direct the expression of the fusion protein, fragments thereof, or functional equivalents thereof, in appropriate host cells. The fusion polypeptide products encoded by such polynucleotides may be altered by molecular manipulation of the coding sequence.

Due to the inherent degeneracy of the genetic code, other DNA sequences which encode substantially the same or a functionally equivalent amino acid sequence, may be used in the practice of the invention for the expression of the fusion polypeptides. Such DNA sequences include those which are capable of hybridizing to the coding sequences or their complements disclosed herein under low, moderate or high stringency conditions described herein.

Altered nucleotide sequences which may be used in accordance with the invention include deletions, additions or substitutions of different nucleotide residues resulting in a sequence that encodes the same or a functionally equivalent gene product. The gene product itself may contain deletions, additions or substitutions of amino acid residues, which result in a silent change.

The nucleotide sequences of the invention may be engineered in order to alter the fusion protein coding sequence for a variety of ends, including but not limited to, alterations which modify processing and expression of the gene product. For example, mutations may be introduced using techniques which are well known in the art, e.g., to insert or delete restriction sites, to alter glycosylation patterns, phosphorylation, to create and/or destroy translation, initiation, and/or termination sequences, or to create variations in coding regions, to facilitate further in vitro modification, etc. One of skill will recognize many ways of generating alterations in a given nucleic acid construct. Such well-known methods include, e.g., site-directed mutagenesis, PCR amplification using degenerate oligonucleotides, exposure of cells containing the nucleic acid to chemical mutagenic agents or radiation, chemical synthesis of a desired oligonucleotide (e.g., in conjunction with ligation and/or cloning to generate large nucleic acids) and other well-known techniques.

Purified fusion proteins may be prepared by culturing suitable host/vector systems to express the recombinant translation products of the DNAs of the present invention, which are then purified from culture media or cell extracts. For example, supernatants from systems which secrete recombinant polypeptide into culture media may be first concentrated using a commercially available protein concentration filter, such as, e.g., an Amicon or Millipore Pellicon ultrafiltration unit. Following the concentration step, the concentrate may be applied to a suitable purification matrix. Affinity chromatography or reverse-phase high performance liquid chromatography (RP-HPLC) may also be used to purify the fusion proteins of the present invention.

Monoclonal or polyclonal antibodies to a purified fusion protein of the present invention may be produced according to conventional methods. For example, described herein are methods for the production of antibodies capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For the production of antibodies to the fusion proteins discussed herein, various host animals may be immunized by injection with the polypeptides, or a portion thereof. Such host animals may include, but are not limited to, rabbits, mice, and rats. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with the polypeptides, or a portion thereof, supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932, 448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

### D. Pharmaceutical Compositions

The natriuretic fusion proteins of the present invention can be administered as pharmaceutical compositions for use in methods to treat or ameliorate pathological conditions in which activation of the NPRA receptor confers a therapeutic benefit on the patient, including but not limited to diseases associated with abnormal diruretic, natriuretic and vasodilatory activity and/or in which it is desirable to induce naturesis, diuresis, vasodilation or to modulate the renin-angiotensis II and aldosterone systems. Such pathological conditions include disorders of the cardiovascular system such as described in detail above. These conditions include those that may be characterized by an excess in extracellular fluid, including but not limited to Chronic Heart Failure (CHF) and pulmonary edema. In a particularly preferred embodiment, the invention includes methods to treat or ameliorate pathological conditions of the cardiovascular system including but not limited to, chronic heart failure (non-ischemic), post-MI heart failure (ischemic CHF), acute MI, reperfusion injury, left ventricular dysfunction (LVD), cardiac fibrosis, diastolic heart failure, and hypertrophic cardiomyopathy. In addition, hypertensive disorders including, but not limited to hypertension, e.g., pulmonary hypertension, systolic hypertension, resistant hypertension and other cardiovascular related diseases such as diabetic nephropathy may be treated or ameliorated by the methods of the present invention. It is also contemplated herein that the fusion proteins and pharmaceutical compositions of the present invention may be provide therapeutic benefit for patients undergoing coronary artery bypass graft procedures.

Pharmaceutical compositions for use in accordance with the present invention may be formulated in a conventional manner using one or more physiologically acceptable carriers or excipients for administration by various means, for example, by inhalation or insufflation (either through the mouth or the nose) or topical, oral, buccal, parenteral or rectal administration. For example, parenteral may include, intradermal, intramuscular, intraperitoneal, intravenous, subcutaneous, intranasal and epidural administration. The compounds may be administered by any convenient route, for example by infusion or bolus injection, by absorption through epithelial or mucocutaneous linings (e.g. oral mucosa, rectal and intestinal mucosa, etc.) and may be administered together with other biologically active agents. Administration can be systemic or local. In a preferred embodiment, it may be desirable to introduce the pharmaceutical compositions of the invention into the affected tissues by any suitable route. Pulmonary administration can also be employed, e.g., by use of an inhaler or nebulizer, and formulation with an aerosolizing agent.

The pharmaceutical compositions may further comprise a vehicle or carrier, including a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, which is herein incorporated by reference in its entirety. The formulation should suit the mode of administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Where appropriate, preparations for oral administration may be suitably formulated to give controlled release of the active compound.

Where appropriate, for buccal administration the compositions may take the form of tablets or lozenges formulated in conventional manner.

In another embodiment, the fusion proteins of the present invention are for administration by inhalation or insufflation (either through the mouth or the nose). As such, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In a particularly preferred embodiment, the pharmaceutical compositions of the present invention may be formulated for parenteral administration by injection, e.g., by bolus injection or continuous infusion. In addition, as contemplated herein, the fusion proteins and pharmaceutical compositions of the present invention may be suitable for self-injection by a patient in need thereof, e.g. long term treatment of CHF. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Alternatively, the active ingredient may be in powder form for constitution with a suitable vehicle, e.g., sterile pyrogen-free water, before use. For example, lyophilized protein compositions may be inhaled or reconstituted then injected in a suitable vehicle.

The compounds may also be formulated in rectal compositions such as suppositories or retention enemas, e.g., containing conventional suppository bases such as cocoa butter or other glycerides.

In addition to the formulations described previously, which may exhibit pharmacokinetics similar to a slow release formulation, the compounds may also be formulated as an actual depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art. For example, for any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC₅₀ (i.e., the concentration of the test compound that achieves a half-maximal inhibition of symptoms). Such information can then be used to determine useful doses and routes for administration in humans.

A therapeutically effective dose or “effective amount” refers to that amount of active ingredient that is nontoxic but sufficient to provide the desired therapeutic effect. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

The present invention further provides kits for use with any of the above methods. Such kits typically comprise two or more components necessary for performing a method described herein. Components may be compounds, reagents, containers and/or equipment. For example, one container within a kit may contain a pharmaceutical composition comprising fusion proteins of the present invention. One or more additional containers may enclose elements, such as reagents or buffers, or equipment to be used in a method to administer the pharmaceutical composition.

It is also contemplated herein that the fusion proteins and pharmaceutical compositions of the present invention may be administered alone or in combination with other compounds or substances that may be used to treat any of the pathological conditions described herein. Such compounds or formulations that may be used in combination therapy with the present invention include, for example, diuretics, beta blockers, and Ang II receptor blockers.

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Although any methods and materials similar or equivalent to those described herein can be used in the practice or testing of the present invention, the preferred methods, devices and materials are now described. All references described herein are expressly incorporated by reference.

### Examples

The materials and methods relevant to the examples described herein are provided immediately below:

### Structural Modeling of the ANP-Fc Fusion/NPRA Interactions:

Monomer Fc Fusion (1 ANP linked, 1 ANP bound): The linker requirements for the ANP and Fc fusions were determined through structure-based modeling using the NPRA and Fc crystal structures (RCSB PDB database 3D crystal structure numbers, 1T34 (co-structure with ANP) and 1HZH, respectively, H. M. Berman, J. Westbrook, Z. Feng, G. Gilliland, T. N. Bhat, H. Weissig, I. N. Shindyalov, P. E. Bourne: The Protein Data Bank. Nucleic Acids Research, 28 pp. 235-242 (2000)). These structures are positioned in space (a) with the C-terminus of ANP and the N-terminus of Fc proximal to one another, yet with enough space between them to minimize steric and electrostatic repulsions, and (b) distal to the two F425 residues (F457 in human full-length NPRA sequence) of the dimeric NPRA to minimize possible interactions with the residues that lead toward NPRA's transmembrane region, which is predicted to start with V474. Once the position of the two structures was finalized, the distance between the C-terminus of ANP and the N-terminus of Fc was measured; this value was divided by 3.5 and rounded up to the nearest whole number to give the minimum number of amino acids (aa) predicted to be needed for the “Fc-linker-hANP28” or “hANP28-linker-Fc” (i.e., Orientation #1 or Orientation #2, respectively, supra pp. 19) fusion to be a viable ligand of NPRA (example: distance=12 A. Minimum linker length=12/3.5=3.4=>4 aa linker). The 3.5 value represents the average amino acid span in β-sheets (the distance from a nitrogen atom in one amino acid to the nitrogen atom in the adjacent amino acid).

Dimer Fc Fusion (2 ANP linked, 1 ANP bound): The procedure outlined for the Monomer Fc Fusion was followed with the added challenge of growing a second linker from the second N-terminus on the Fc toward one ANP-NPRA trimeric complex and one ANP peptide in its binding conformation as defined in 1T34, RCSB PDB Database 3D Structure (H. M. Berman, J. Westbrook, Z. Feng, G. Gilliland, T. N. Bhat, H. Weissig, I. N. Shindyalov, P. E. Bourne: The Protein Data Bank. Nucleic Acids Research, 28 pp. 235-242 (2000)).

Dimer Fc Fusion (2 ANP linked, 2 ANP bound): The procedure outlined for the Monomer Fc Fusion was followed with the added challenge of growing a second linker from the second N-terminus on the Fc toward two ANP-NPRA trimeric complexes.

### Cloning, Expression and Purification of CysFc:

The vector pEE12,4 was licensed from Lonza group (Basel, Switzerland). The human Fc sequence from IgG1 was obtained from a human leukocyte cDNA library with the mouse Ig_{K} signal sequence (in bold below) using standard PCR techniques (pSYNFc-001, pSYNFc-007). The cysFc sequence with the mouse Ig_{K} signal sequence was obtained from pSYNFc-007 (pSYNcysFc-002). The CysFc sequence was then cloned into pEE12.4 through an intermediate cloning step (pSYNcysFc-003) to create pSYNcysFc-004. CysFc-004 has the following amino acid sequence:

(SEQ ID NO: 18)
METDTLLLWVLLLWVPGSTGCPPCPAPELLGGPSVFLFPPKPKDTL
MISRTPEVTCVVVDSHEDPEVKFNWYVDGVEVHNAKTKPREEQYNS
TYRVVSVLTVLHQDWLNGKEYKCKVSNKALPAPIEKTISKAKGQPR
EPQVYTLPPSRDELTKNQVSLTCVKGFYPSDIAVEWESNGQPENNY
KTTPPVLDSDGSFFLYSKLTVDKSRWQQGNFSCSVMHEALHNHYTQ
KSLSLSPGK.

Lonza CHOK1Sv suspension cells (1×10⁷ cells) were transfected with 40 μg of Pst1 linearized pSYNcysFc-004 by electroporation according to the manufacturer's instructions. Cells were plated in 40×96 well plates at a seeding density of 2500 cells/well. Twenty four hours later selection was initiated with 25 μM MSZ and cells were incubated at 37° C./5% CO₂ for three weeks. Transfected cells were expanded from 96 well plates to 24 well plates to T25 and T75 flasks before adapting to serum-free suspension culture. CysFc-004 (CysFc) expression levels were monitored during growth curves in serum-free suspension using a protein A immunoprecipitation procedure followed by non-reducing SDS-PAGE analysis. Based on these growth curves, the cell line 9F4 was chosen for production.

Cys-Fc-004 (CysFc) was produced from 20 L of 9F4 cells seeded at 5×10⁵ cells/ml in 3 L Fernbach flasks (1 L cells/flask). At the end of the production run, cells were removed by centrifugation and the conditioned medium concentrated 4-fold by tangential flow filtration. CysFc-004 was purified by protein A chromatography, the column washed with Dulbeccos PBS (DPBS) followed by DPBS containing 0.9 M NaCl. The protein was eluted with 0.1 M sodium citrate containing 0.15 M sodium chloride, pH 3.4. The protein was then diafiltered into DPBS and stored at 4° C. until use.

### Synthesis of C-Terminal ANP Thioesters:

ANP peptide (0.1 mmol) was synthesized on an Advanced ChemTech 3960 synthesiser on Fmoc-Gly-NovaSyn® TGT (Novabiochem) by standard Fmoc-solid phase peptide synthesis using HBTU as the coupling agent, N,N-diisopropylethylamine (DIEA) as the base and N,N,-dimethylformamide (DMF) as the solvent. After automated synthesis, the N-terminus was protected by manually treating the resin with 0.218 g (10 eq.) of the di-t-butyl dicarbonate (Boc₂O) and 174 μL (20 eq.) of DIEA in 10 mL DMF, and allowed to mix overnight at room temperature. The reaction was vacuum-filtered, and the resin was washed with DMF and dichloromethane (DCM). The fully protected peptide was cleaved from the resin by mild acid cleavage in DCM with 1, 1, 1, 3, 3, 3-hexafluoro-2 propanal (HFIP), in 50 mL of 3:7 HFIP:DCM for two hours. The resin was filtered into a pre-tared round bottom flask, and the filtrate was concentrated in vacuo over-night, yielding the protected peptide with a free C-terminus. To generate the thioester, this material was treated with 1.5 eq. of glycine benzyl thioester hydrochloride salt (Gly(SBn) HCl), 1.5 eq. of PyBOP (Novabiochem, and 4.5 eq. of DIEA in 10 mL DMF and stirred for 18 hours. The reaction mixture was again concentrated in vacuo for 18 hours. The protected peptide thioester was deprotected by treatment with 25 mL of cleavage cocktail (23.75 mL trifluoroacetic acid (TFA), 0.625 mL triisopropysilane (TIS), 0.625 mL ethanedithiol (EDT)) for two hours and 45 minutes, after which 0.625 mL bromotrimethysilane (TMSBr) was added and allowed to mix for 15 minutes. The reaction mixture was concentrated, and the crude peptide thioester was precipitated with cold diethyl ether (Et₂O). Peptide was centrifuged, supernatant decanted, and crude peptide was triturated two more times with cold Et₂O. The crude peptide was then purified by reverse phase prepatory HPLC (Phenomenex Jupiter 5u C4 300 A column, 250×21.20 mm); HPLC method parameters are listed below. Fractions were analyzed by LC/MS (Applied Biosystems, Mariner) for identity and fractions containing >90% peptide were pooled, flash frozen, and lyophilized yielding the pure ANP C-terminal thioester.

### Synthesis of N-Terminal ANP Thioesters:

ANP peptide (0.1 mmol) was synthesised on an Advanced ChemTech 396Ω synthesiser on Fmoc-Tyr(tBu)-NovaSyn® TGT (Novabiochem) by standard Fmoc-solid phase peptide synthesis using HBTU as the coupling agent DIEA as the base and DMF as the solvent. After automated synthesis, the N-terminus was converted to a free carboxylic acid by manually treating the resin with 0.100 g succinic anhydride (10 eq.) and 174 μL (10 eq.) DIEA in 10 mL DMF for two hours at room temperature. Reaction contents were removed via vacuum filtration, and the resin was washed with DMF and DCM respectively. To generate the thioester, this material was treated with 0.109 g (5 eq.) of Gly(SBn).HCl, 0.260 g (5 eq.) of PyBOP (Novabiochem), and 261 μL (15 eq.) of DIEA in 10 mL DMF and stirred for 18 hours at room temperature. The reaction contents were then removed via vacuum filtration, and the resin was washed with DMF and DCM. The protected peptide thioester was cleaved from the resin and deprotected by treatment with 25 mL of cleavage cocktail (23.75 mL TFA, 0.625 mL TIS, 0.625 mL EDT) for two hours and 45 minutes, after which 0.625 mL TMSBr was added and allowed to mix for 15 minutes. The reaction contents were filtered, conserving the filtrate. The filtrate was concentrated, and crude peptide thioester was precipitated with cold diethyl ether (Et₂O). The peptide was centrifuged, the supernatant was decanted, and crude peptide was triturated two more times with cold Et₂O. The crude peptide was then purified by reverse phase preparatory HPLC, as previously described. Fractions were analyzed by LC/MS (Applied Biosystems, Mariner) for identity and fractions containing >90% peptide were pooled, flash frozen, and lyophilized yielding the pure ANP C-terminal thioester. Structures/sequences and mass spectral data are shown below and in Table 1.

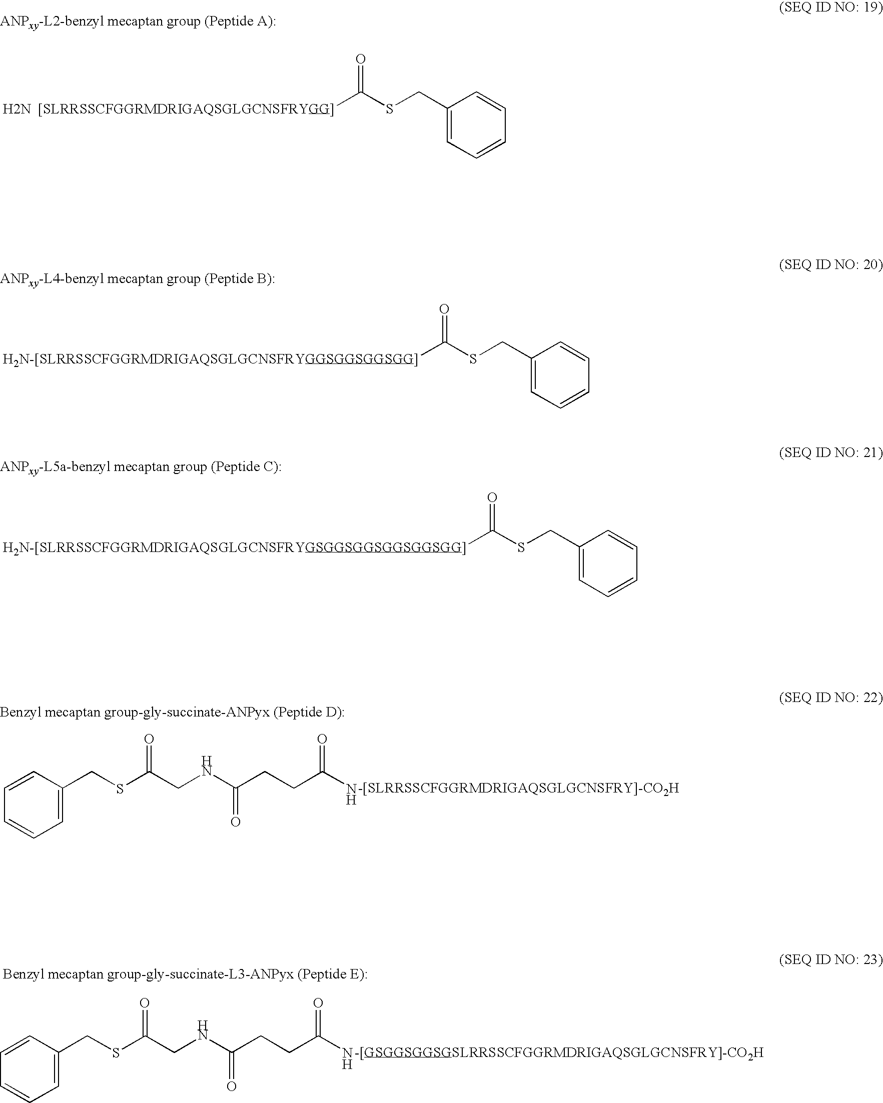

TABLE 1
Mass spectral data.

| | | |
|---|---|---|
| Peptide | MW_{calculated} (Da) | MW_{observed}* (Da) |

| | | | |
|---|---|---|---|
| D | 3345.84 | 3345.28 | (M + 1) |
| A | 3302.82 | 3302.29 | (M + 1) |
| | | 3417.12 | (M + TFA) |
| B | 3906.37 | 3905.31 | (M + 1) |
| | | 4020.14 | (M + TFA) |
| E | 3949.39 | 3950.14 | (M + 1) |
| C | 4251.68 | 4251.58 | (M + 1) |
| | | 4365.54 | (M + TFA) |

*As determined by LCMS from a single ion conversion process of the multiply-charged peptide ions

### Conjugation of ANP Thioester Peptides to Cys-Fc:

ANP peptide S-benzyl thioesters were conjugated to the N-terminus of Cys-Fc. Cys-Fc (5 mg/mL in PBS, pH 7.4) was treated with 2-mercaptoethanesulfonic acid, sodium salt (MESNA, Sigma-Aldrich) such that the final concentration of MESNA was 20 mM. Peptide thioester was added to the reaction mixture (2-6 mol equivalents depending on the peptide) and allowed to gently mix for 18 hours at room temperature. The actual number of equivalents of peptide was selected based on pilot scale reactions such that equal percentages of monomer (1 ANP peptide per Fc) and dimer (2 ANP peptides per Fc) conjugate were formed. The crude reaction mixture was diluted to 1 mg/mL in PBS and dialyzed exhaustively against PBS (pH 7.4) (7 changes over 24 hours). NuPage SDS-PAGE gels (Invitrogen) were used to determine the extent of reaction. Prior to protein purification, the conjugate was dialyzed into 50 mM Tris-HCl buffer pH 7.2.

### Purification of the Semi-Synthetic ANP-Fc Conjugates:

The dialyzed ANP-Fc conjugation reaction mixture was adjusted to a final concentration of 50 mM Sodium Acetate, pH 5.0 with 1M Sodium Acetate pH 5.0 then filtered through a 0.8/0.2 μM syringe filter. This clarified solution was loaded onto a 1×10 cm column packed with Fractoprep SO₃-650(M) cation exchange resin (CEX) equilibrated with 50 mM Sodium Acetate, pH 5.5. After loading, the column was washed with 3 column volumes (CV) of equilibration buffer. The protein was eluted with a linear gradient from 0M-0.5M Sodium Chloride (in 50 mM Sodium Acetate pH 5.5) over 30 CV. Fractions were analyzed using NuPage SDS-PAGE gels (Invitrogen) and the fractions containing the majority of the monomer conjugate were pooled together, while separately the fractions containing the majority of the dimer conjugate were pooled together. The CEX pools were first adjusted to 0.1M Tris pH 7.2 using a 1M Tris pH 7.2 stock solution, then adjusted to 1M Ammonium Sulfate using a 3M Ammonium Sulfate stock solution. After filtration with a 0.8/0.2 μM syringe filter this clarified protein solution was loaded onto a 1×10 cm column packed with EMD Fractogel TA 650(S) thio-affinity resin pre-equilibrated with 50 mM Tris, 1 M Ammonium Sulfate, pH 7.2. After washing the column with 3 CV of equilibration buffer the protein was eluted with a decreasing linear gradient from 1.0M to 0.33M Ammonium Sulfate over 20 CV. This was followed by a step gradient from 0.33M to 0M Ammonium Sulfate. The peak fractions containing highly purified conjugate were identified using SDS-PAGE gels then pooled together. The final pool was dialyzed against 1× Dulbecco's Phosphate Buffered Saline (1 Liter×2 Changes) (Invitrogen). Protein concentration was determined using UV-280 nm analysis. If needed, the final protein was concentrated using Amicon Ultra-15 Centrifugal Concentration Unit(s) (Millipore). The final protein was aseptically filtered through a 0.2 μM filter then aliquots are stored at −80° C.

### Size-Exclusion Chromatography of the Semi-Synthetic ANP-Fc Fusions:

The aggregation profile of the semi-synthetic ANP-Fc fusion proteins was assessed using analytical size exclusion chromatography (SEC-HPLC). The TSKgel Super SW2000 4.6 mm (Tosoh Biosciences) SEC-HPLC column was equilibrated with PBS, pH 7.4+250 mM NaCl. Area under the curve integration was done to estimate the percent of total protein in each peak.

For example, a TSKgel Super SW2000 4.6 mm (Tosoh Biosciences) SEC-HPLC column was run at a flow rate of 0.4 mL/min using PBS, pH 7.4 and 250 mM NaCl as the eluent. 5 μL of a 1.0 mg/mL sample was injected for each analysis. BioRad MW standards (cat#151-1901) were injected prior and after each set of injections to ensure the integrity of the column.

### Characterization of “Free” Peptide in the Semi-Synthetic ANP-Fc Fusions:

The “free” unconjugated ANP peptide contaminant levels in the purified semi-synthetic ANP-Fc fusions preps was determined using reversed-phase chromatography. A Protein C4 reversed phase column (Grace Vydac) was injected with 100 μg of the purified semi-synthetic Fc fusion protein. The two mobile phases were A) 0.1% TFA in water and B) 0.1% TFA in Acetonitrile. The reversed phase chromatography was run using the following gradient profile over a total run time of 50 minutes (min): 0.5 min at 5% B, 5-35 min ramp up to 95% B, 35-40 min hold at 95% B, 40-42 min ramp down to 5% B, 42-50 min hold at 5% B. Alternate runs were spiked with 0.1 μg of “free” ANP peptide (0.1 μg spike is equivalent to 1.3 mole %) to visualize expected positioning and peak heights of potential contamination. The lower limits of “free” ANP detection of this method are to 1% molar ANP contamination levels.

### Characterization of Peptide Disulfide Linkages in the Semi-Synthetic ANP-Fc Fusions:

Trypsin digestion of ANP-Fc fusions was performed at pH 5.25, 37° C. for 18 hours, preserving the disulfide (Cys-Cys) linkages. The tryptic digest was split into two aliquots, one of which was reduced by DTT and capped with 2-iodoacetamide. The reduced sample (0.5-1.0 pmol) was loaded onto a self-packed C18 capillary column (10 cm) hooked to a nano-LC/MS/MS. Peptide sequencing was performed using Sequest or de novo. Free peptide (oxidized form) was treated the same way (reduced and non-reduced) and used as a positive control.

An Agilent 1100 HPLC binary system and a Thermo LCQ-Advantage ion trap mass spectrometer was used for the LC/MS/MS analysis. The unconjugated ANP peptide analog (including GS linker region) of the ANP-Fc fusions was used as the positive control for the identification experiment. 3 μg of peptide-Fc fusion (1 μg of free peptide in oxidized form) was diluted to a final volume of 30 μL with 100 mM ammonium acetate (pH 5.25). 10 μL of a 0.01 μg/mL trypsin solution (trypsin gold, MS grade, Promega) was added to the protein/peptide solution and the digestion is carried out at 37° C. for 18 hours. The digested sample was split into two aliquots of 20 μL. One aliquot of the digest (non-reduced condition) is frozen immediately and stored at −20° C. until analysis. The 2^{nd} aliquot is dried down in vacuo and redissolved with 20 μL of 50 mM Tris-HCl and 1 mM EDTA (pH 8.0). One microliter of DTT (0.2 M) is added and the solution is incubated at 56° C. for 30 minutes. Subsequently, one microliter of 2-iodoacetamide (0.5 M) is added and the reaction proceeded for 30 minutes in the dark to generate the thiol-capped digestion sample. The unconjugated peptide digest is diluted five-fold with water. 0.5 μL of digested peptide-Fc fusion (or 1 μL of digested peptide) is loaded onto a self-packed C 18 capillary LC column (YMC C 18, 5 μm, 10 cm long) for nano-LC/MS/MS analysis. The HPLC pump flowrate of 0.2 mL/min is split to obtain 1 μL/min for the capillary LC. The mobile phases used is 0.05 M acetic acid in water (A) and 0.05 M acetic acid in acetonitrile (B). The LC/MS data (non-reduced and reduced) are compared to theoretical masses corresponding to possible disulfide folding linkages for the digested fragments to determine whether or not the proper disulfides within the ANP peptide(s) are formed. The reduced peptide fragment sequence is confirmed by SEQUEST database searching of MS/MS data.

### Characterization of the FcRn Binding Properties of the Semi-Synthetic ANP-Fc Fusions:

Purified soluble human FcRn was bound to Biacore chip surfaces at pH=6.0. The semi-synthetic ANP-Fc fusions were flowed over the chip surfaces in a series of different concentrations. The equilibrium responses (R_{eq}) were fit by non-linear regression to a model with two classes of non-interacting binding sites using the Biacore BiaEval software. This modeling assumes there are two classes of Fc binding sites on the FcRn coated sensor chip (K_{D1}, K_{D2}) that contribute to the overall observed binding.

For example, soluble human FcRn were cross-linked to the dextran surface of a CM5 sensor chip by amine coupling using 1-ethyl-(3-dimethylaminopropyl)-carbodiimide hydrochloride (EDC) and N-hydroxysuccinimide (NHS) as recommended by Biacore (BIAapplications Handbook, version AB, section 4.2). For immobilization, the FcRn was diluted to 10 μg/mL in 10 mM sodium acetate, pH 4.5. Residual sites on the dextran were blocked with 1 M ethanolamine hydrochloride pH 8.5. FcRn was immobilized to one flow cell on the sensor chip, while a control flow cell was blocked with ethanolamine for reference subtraction. For analysis of ANP-Fc conjugates, FcRn was coated to a final density of 400 to 600 response units (RU). This level of coating was used to ensure that the sensor chip was fully saturated by the injected Fc protein within the allowable injection time while still giving a reasonable signal. Experiments were performed at pH 6 using 50 mM sodium phosphate, 100 mM sodium chloride, 0.01% surfactant P20 (Biacore AB). Eleven—2 fold serial dilutions between 1 μM and 1 nM of the ANP-Fc or control proteins were injected over the FcRn-CM5 chip at 30 μL/min for 5 minutes. Bound protein was then dissociated from the chip for 2.5 minutes with running buffer. Any remaining protein was removed from the chip with a 27 second injection of pH 7.4 HBS-P buffer (0.01 M HEPES pH 7.4, 0.15 M NaCl, 0.005% v/v Surfactant P20) at 30 μL/min.

Next, sensorgrams were analyzed using BiaEval software version 3.1 (Biacore AB) and were baseline corrected using a buffer blank, then baseline averaged. When a given concentration of the protein was injected onto the chip, the signal gradually reached a maximal plateau. This was presumed to be the extent of binding at equilibrium and the observed signal was termed Req (Response Units at Equilibrium). Req values were plotted against concentration and the equilibrium affinity constants (K_{D}'s) were then derived by fitting the data to a heterogeneous ligand model using BiaEval software. This model assumes that there are two classes of non-interacting binding sites of FcRn on the sensor chip. Thus, the K_{D} values were reported as two separate Kg's that contribute some fractional % to the theoretical maximal observed Req value (Rmax).

### Recombinant ANP-Fc Fusion Protein Production and Characterization:

ANP-Fc Fusion Construct Generation: Four DNA sequences were synthesized according to conventional oligonucleotide synthesis techniques, and subcloned into the vector pJ13 (DNA2.0 Inc.). Each construct contained the same basic order of a HindIII restriction enzyme (RE) site followed by a Kozak sequence, mouse IgG kappa light chain signal sequence, human ANP28 sequence, a linker sequence, a human Fc gamma sequence followed by another RE site. The Kozak sequence and the mouse IgG kappa light chain signal sequence were identical to the sequence in the vector pSYN-Fc-015 (pcDNA3.1/Igk sig seq-hFc). The pSYN-Fc-015 vector expresses the Fc protein of an IgG1 isotype protein that matches the sequence in NCBI database for accession number Y_{—}14735. The human ANP28 sequence is from the NCBI database with the accession number NM_{—}006172 (SEQ ID NO. 15). The linker sequence encodes for either an 11 or 20 aa peptide consisting of repeats of glycine and serine residues, (GGS)₃GG (SEQ ID NO: 3) for the 11 aa linker (L4) and (GGS)₆GG (SEQ ID NO: 7) for the 20 aa linker (L6). Constructs pJ13-ANP28-11aa-hFc1 and pJ13-ANP28-20aa-hFc1 contain sequence identical to the first 39 bp of the human Fc gamma 1 sequence in pSYN-Fc-015 which includes a BspEI RE site. Constructs pJ13-ANP28-11aa-hFc2 and pJ13-ANP28-20aa-hFc2 are the same as the two previously described constructs through the linker sequence except the entire human Fc gamma 1 sequence is replaced with a human Fc gamma 2 sequence. Following the human Fc2 sequence an EcoRI restriction site is present.

The expression vector pSYN-Fc-015 was digested with HindIII and EcoRI and the fragment containing the Kozak sequence, mouse IgG kappa light chain signal sequence and the entire Fc gamma 1 sequence was removed. The 5.4 kb vector backbone was isolated by standard molecular biology techniques. Constructs pJ13-ANP28-11aa-hFc2 and pJ13-ANP28-20aa-hFc2 were each digested with HindIII and EcoRI; an 858bp fragment was isolated from pJ13-ANP28-11aa-hFc2 and an 885bp fragment was isolated from pJ13-ANP28-20aa-hFc2 using standard molecular biology techniques. Each contained the Kozak sequence, mouse IgG kappa light chain signal sequence and either an 11aa linker (L4) or a 20aa linker (L6) followed by the entire Fc gamma 2 sequence. The 5.4 kb vector was ligated to either the 858 bp fragment or the 885 bp fragment overnight with T4 DNA ligase at 4° C. The ligated DNAs were transformed into the bacterial strain DH5a, plated onto LB+ampicillin plates and incubated at 37° C. overnight. The following morning the plates were removed from the incubator and several single colonies from each ligation/transformation plate were each picked into 15 ml of LB media containing ampicillin and grown overnight at 37° C. in a shaking incubator. The next day the bacterial cells were harvested and plasmid DNA was isolated using QIAprep Spin Miniprep Kit (Qiagen Inc.). Aliquots were sent out for sequencing to confirm the DNA sequence (Agencourt).

Vector pSYN-Fc-015 was digested with HindIII and BspEI and the 6 kb vector backbone was isolated. This removed the Kozak sequence, mouse IgG kappa light chain signal sequence and the first 37 bp of the Fc gamma 1 sequence. Constructs pJ13-ANP28-11aa-hFc1 and pJ13-ANP28-20aa-hFc1 were digested with HindIII and BspEI; a 209 bp fragment was isolated from pJ13-ANP28-11aa-hFc1 and a 236 bp fragment was isolated from pJ13-ANP28-20aa-hFc1. Each fragment contained the Kozak sequence, mouse IgG kappa light chain signal sequence and either an 11aa linker (L4) or a 20aa linker (L6) as well as the first 37 bp of the Fc gamma 1 sequence. The 6 kb vector was ligated to either the 209 bp fragment or the 236 bp fragment; transformations, DNA plasmid preps and sequencing were performed as previously described.

Mammalian Expression of the ANP-Fc Fusions: The initial expression of the ANP fusion constructs was done in HEK293 cell lines using a transient transfection protocol on a 1 L scale. HEK293 cells were grown in FreeStyle 293 medium and transfected using the 293Fectin reagents and protocols as described by the manufacturer (Invitrogen). Transiently transfected cells were grown in a spinner flask with agitation of 90 rpm at 37° C. and 5% CO₂. The cells were monitored daily for cell density, viability, and diameter, level of glucose, lactate, glutamine and pH. The conditioned media was harvested 72 hours after the transfection by centrifugation at 3600 rpm for 15 minutes and was delivered fresh to purification. Expression levels were determined by SDS-Page and Western-blotting analysis.

Once the expression levels were determined for each construct, various scale-up methods, including 20 L transient transfection and stable pool generation, were performed to generate the protein required. The 20 L transient transfection was carried out using the PEI-25 reagent in a 50 L wave bioreactor. A 25 mg aliquot of DNA was diluted into 250 ml of FreeStyle 293 media, and 75 mg aliquot of PEI-25 was diluted into 250 ml of the same media. The DNA sample was then mixed with PEI solution. After a 15 minute incubation at room temperature, the DNA:PEI mixture was loaded into the wave bioreactor with 10 L of freshly loaded HEK293 cells at the cell density of 2 million/ml. The wave bioreactor was set at a rocking angle of 8 degrees and a rocking speed of 22 rpm at 37° C. and 5% CO₂. Five hours after the transfection, 10 L of FreeStyle 293 medium was loaded into the wave bioreactor. The cells were monitored daily for cell density, viability, and diameter, level of glucose, lactate, glutamine and pH. The conditioned media was harvested 120 hours after the transfection by centrifugation at 3600 rpm for 15 minutes and was delivered fresh to purification. Expression levels were determined by SDS-Page and Western-blotting analysis. The stable pools generation started in a flask where stable cells were selected with 400 μg/ml of G418 and then transferred into spinner. Once the cells stabilized under these conditions, they were expanded and 4-5 L runs were carried out either in spinners or a wave bioreactor.

Purification of the Recombinant ANP-Fc Fusion Proteins: The purifications were performed at 4° C. using an AKTA system (GE Healthcare) or BioOptix10 (ISCO). Five milliliters of Protease Inhibitor Cocktail (Sigma) were added per liter of conditioned media. A column was packed with 3-4 mL Protein A Ceramic HyperDF® resin (Pall) per liter of conditioned media and equilibrated with 10 CV of Equilibration Buffer (1× Dulbecco's PBS without Mg/Ca, pH 7.3, Invitrogen) at 1 mL/min. Conditioned media was loaded onto the column overnight at 1-5 mL/min depending upon the volume of media. The resin was then washed, in 10 CV sequences, with Wash Buffer 1 (DPBS without Mg/Ca, pH 7.3), Wash Buffer 2 (DPBS without Mg/Ca pH 7.3+1M NaCl), and then Wash Buffer 1 again. The bound protein was eluted with 10 CV Elution Buffer (0.1 M Glycine/HCl, pH 2.5 in dH₂O) at 2.5 ml/min. The 3 mL fractions were neutralized immediately with 300 μL (10% of fraction) of Neutralization Buffer (1M Tris-HCl pH 8.0, Invitrogen). The fraction pools were dialyzed against PBS (Hyclone), 2×5 L for total of 22 hours, using Slide-A-Lyzer cassettes (Pierce) and concentrated using Amicon Ultra-15 10 kDa MWCO concentrators (Millipore) to 1-2 mg/mL. Aliquots were flash-frozen in liquid nitrogen and then stored at −80° C. Final products were characterized by SDS-PAGE (non-reduced and reduced), analytical ultracentrifugation and mass spectroscopic analysis before being submitted for biological function assays. Endotoxin contaminant levels were determined using the gel-clot LAL reagents (The Associates of Cape Cod).

Analytical Ultracentrifugation of the Recombinant ANP-Fc Fusion Proteins: Sedimentation velocity experiments were conducted to assess the purity and aggregate content of the purified ANP-Fc fusions. The ANP-Fc fusions were evaluated by sedimentation velocity in a phosphate buffered saline buffer containing 10 mM Sodium Phosphate, 150 mM NaCl, pH 7.3. Samples were loaded into centrifuge cells containing double-sector charcoal-epon centerpieces and quartz windows. Data was collected using a Beckman XLI analytical ultracentrifuge at 280 nm, 50,000 rpm and 20° C. Solution densities and viscosities were measured using an automated Anton-Paar AMVn/SP3-V viscometer and DMA4500/DMA5000 densitometer at 20° C. The sedimentation data were analyzed using the program SEDFIT version 9.3b (2).

Mass Spectroscopy Analysis of the Recombinantly Produced ANP-Fc Fusions: Mass spectrometric experiments were conducted to measure intact molecular weight, to perform partial sequence validation via enzymatic digestion and peptide mapping, and to identify degradation products of the ANP-Fc fusions.

To evaluate intact molecular weight 5 μl of fusion protein was injected onto a Jupiter C4 column (5 u, 300 A, 1.0×150 mm) at a flow rate of 40 μl/min. The protein is eluted with a gradient from 0% to 100% A in 40 minutes [Mobile phase A is 98/2/0.06 (Water/Acetonitrile/TFA) and Mobile phase B is 95/5/0.052 (Acetonitrile/Water/TFA)]. The effluent was flowed into a Micromass Quattro Ultima mass spectrometer scanning from 900-1500 amu with a 2 second scan time. Data was deconvoluted using the program MaxEnt1 to yield molecular weight(s).

The fusion proteins were digested with trypsin or Endo-LysC (8010-108) for peptide mapping and degradation analysis. Briefly, 25 μl of each sample was denatured, reduced, and alkylated. Trypsin or Endo-LysC was added, and the sample incubated overnight in a 37° C. water bath. Analysis was performed via HPLC/FTMS. The instrument generated multiple scans during the chromatographic separation at approximately 50,000 resolution. The mass of each eluting peptide was determined with an accuracy of 0.0005% relative error. Data was processed with the ProQual program (3) to yield the peptide map. Peptides predicted based on enzyme specificity were matched if the theoretical value was within 0.0005% of the experimentally determined value. The Endo-LysC data was also searched for N-terminal truncations by looking for the masses corresponding to possible amino acid deletions. Unlike trypsin, the Endo-LysC experiment allows for observation of the intact amino terminal region.

### Generation of Stable Cell Lines for In Vitro NPRA Cell Assays:

Full-length human NPRA, human NPRB, and human GUCY2C (NPRC) sequence containing plasmids were purchased from OriGene Technologies, Inc. (Rockville, Md.) then sub-cloned into pcDNA3.1 directional mammalian expression vector (Invitrogen). Insert orientation and nucleotide sequence of each construct was verified by an outside vendor (SeqWright, Inc.). The pcDNA3.1 NPR clones were transfected using Lipofectamine (Invitrogen) into HEK293 cells where stable cell lines expressing human NPRA, human NPRB, or human NPRC are selected using G418. Clones were screened using the natriuretic peptide induced cGMP assay described below. (NPRA clones were treated with human ANP while NPRB clones were treated with human CNP (Sigma)). High cGMP producing clones were expanded. NPRC clones were screened using a ¹²⁵I ANP binding assay. ¹²⁵I ANP high binders were expanded. Cell lines were grown in DMEM containing, 100 μg/ml penicillin/streptomycin, L-glutamine, 400 μg/ml of G418, and 10% FBS (Hyclone)). HEK293T-GCA (rat NPRA expressing cells) (Lincoln Potter, Dept. Biochemistry, Molecular Biology and Biophysics University of Minnesota, Minneapolis/St. Paul, Minn.) were grown in DMEM containing 100 μg/ml penicillin/streptomycin, L-glutamine, Hygromycin B and 10% FBS.

Natriuretic Peptide Fusion Protein Induced cGMP Assay:

Natriuretic peptides of >95% HPLC purity were obtained from Sigma. HEK293 NPRA cells grown to 90% confluence were harvested using HANKS based Cell Dissociation Medium (GIBCO). Cells were washed and resuspended at 3.3×10⁵ cells per mL in pre warmed Dulbecco's PBS, pH 7.4, 25 mM HEPES, 0.1% BSA, 500 μM 3-Isobutyl-1-methylxanthine (IBMX) [Assay Buffer]. Assays were performed in Optiplate-96 White Opaque 96-well Microplates (Perkin Elmer). 15 μL of cell suspension is added to 15 μL of 2× fusion protein in Assay Buffer in triplicate and incubated for 20 minutes at 37° C. The cGMP concentration was measured using the HitHunter™ cGMP Assay Kit (DiscoveRx Corporation). cGMP production dose response curves were generated with a four parameter logistic equation fitted using the Levenburg Marquardt algorithm in XLfit4.2 data analysis software (ID Business Solutions, Ltd.).

### ¹²⁵I ANP Binding Assay:

Millipore Multiscreen glass fiber filter microtiter plates were coated with 0.2% polyethylenimine (PEI) (Sigma) and washed with binding buffer (RPMI, 0.1% BSA) by vacuum filtration. 100 ml of 1*10⁶ HEK293-NPRC cell clone suspension in binding buffer were added to each well. 100 ml of 2× ¹²⁵I-ANP+/−1000× cold ANP (non-specific binding control) were added to the appropriate wells. The plates were incubated on an orbital shaker at room temperature for two hours. Following the incubation, the plates were washed 5× with 200 μl of cold binding buffer followed by a 200 μl final wash with Wash Buffer (10 mM Tris, 200 mM NaCl, 0.02% BSA) using a vacuum manifold. The filters were allowed to dry at room temperature. 30 μl of Microscint 20 was added to each well. The plates were read on a PerkinElmer Topcount NTX Liquid Scintillation Counter.

### Pharamacokinetics of ANP-Fc in Rats:

Femal Wistar rats (˜100 g; 4 rats/protein) were dosed intravenously with 0.5 mg/kg monomer (Construct 14) and dimer (Construct 15) or monomer (Construct 10) and dimer Construct 11) in PBS. Blood was collected from each rat by tail nick at 1, 2, 4, 8, 24, 48, and 72 hours (Construct 15) or 0.25, 1, 2, 4, 8, 24, 48, and 72 hours (Constructs 10, 11 and 14) after dosing. Blood (2×60 μl aliquots) was collected into microcapillary tubes (Fisher Scientific, cat #22-362-574) containing 6 μl 3.2% sodium citrate and plasma generated by centrifugation. Plasma was stored at −20° C. until analysis by ELISA.

### Oral Uptake of ANP-Fc in Neonatal Rats:

Ten day old neonatal Wistar rats (4 rats/group) were orally dosed with 0.5 mg/kg ANP-Fc dimer (Construct 14) in PBS supplemented with 5 mg/ml soybean trypsin inhibtor (Calbiochem; catalog #650357). Blood was collected from a group of rats by cardiac puncture at 1, 2, 4, 8, 24, 48, and 72 hours after dosing. Blood (700 μl) was collected into 0.1 volume 3.2% sodium citrate and plasma generated by centrifugation. Plasma was stored at −20° C. until analysis by ELISA.

### ANP/Fc ELISA:

96-well plates (Costar, catalog #369) were coated with 5 μg/ml (50 μl/well) mouse anti-human ANP (US Biologicals, Cat #A4150) in 50 mM of carbonate/bicarbonate buffer pH 9.6 at 4° C. overnight. Plates were blocked with 300 μl/well of PBS containing 5% bovine serum albumin (PBS/5% BSA) (Jackson ImmunoResearch#001-000-162) for 2 hours at room temperature (RT). Standards and samples (100 μl/well) were diluted in PBS/5% BSA and incubated for 2 hours at RT. Standard curves ranged from 0.039 ng/ml to 10 ng/ml. Plates were washed three times with 300 μl/well of PBS containing 0.05% Tween-20 (PBST) in a Tecan plate washer. Plates were then incubated with 100 μl.well of goat anti-human (Fc specific) horseradish peroxidase (HRP) conjugated antibody (Pierce Biotechnology, cat#31414) diluted 1:7,500 in PBS/5% BSA for 3 hours at room temperature. Plates were washed four times with 300 μl/well of PBST before development with 100 μl/well of TMB supersensitive subtrate (BioFx Laboratories, cat#TMBS) at RT for approximately 6 minutes. Reactions were stopped by addition of 0.25M of sulfuric acid (100 μl/well). Plates were read at 450 (−600) nm in a Spectromax plate reader.

### Example 1

### Structural Modeling to Predict Minimum Linker Distance

Structural modeling was done to determine the linker requirements for the Fc and ANP fusions. With the both molecules oriented to minimize distance as well as minimize steric and electrostatic repulsions, a minimal distance from the C-terminus of ANP is 12 Å from the closest N-terminus of the Fc dimer and 17 Å from the other N-terminus of the Fc dimer. If the Fc dimer has only one ANP fused, a 4 to 6 aa minimum linker length would be suggested. With two ANP peptides bound to the Fc dimer, if only one ANP bound (in a 1:1 Fc dimer:NPRA ratio) then a minimum linker length of 9 aa for each linker would be suggested. For both ANP's to bind in a 1:2 Fc dimer:NPRA ratio, linkers with a minimum length of 12 aa would be suggested.

### Example 2

### Synthesis and Characterization of Synthetic ANP Fusion Proteins

Synthetic chemistry was used to generate hANP28 peptides with linkers fused to human Fc of IgG1 isotype. The resulting semi-synthetic ANP-Fc fusion molecules generated are shown in Table 2. The linkers tested were glycine succinate (L1), GlyGly (L2), (GlyGlySer)₃GlyGly (L4) (SEQ ID NO: 3) and Gly(SerGlyGly)₂SerGly (L3) (SEQ ID NO: 2). The glycines (G) were used to add flexibility, while the polar serines (S) were built in to add solubility. The hANP28 peptides with linkers were linked to recombinantly produced Cys-Fc in two orientations: 1) C-terminus of hANP28+Linker fused to N-terminus of Cys-Fc [Orientation #1], and 2) N-terminus of Linker+hANP28 fused to N-terminus of Cys-Fc [Orientation #2]. The synthetic chemistry of the first orientation resulted in a complete peptide bonded structure while the chemistry of the second orientation left a succinate moiety in place of one amino acid of the fusion.

The semi-synthetic ANP-Fc conjugates were purified using a two-step purification protocol consisting of cation exchange chromatography followed by thio-affinity chromatography. The cation exchange step separated the Cys-Fc, the monomer conjugate (1 ANP peptide per Fc), and dimer conjugate (2 ANP peptides per Fc), while also removing the protein aggregates. The individual ANP-Fc conjugate cation exchange pools were independently put through a thio-affinity chromatography purification step to remove the unconjugated free ANP peptide and “polish” the monomer and dimer conjugate species.

The purified semi-synthetic ANP28-Fc fusions were extensively characterized using SEC-HPLC, reverse-phase chromatography, mass spectroscopy, and molecular binding analysis. The SEC-HPLC, used to assess aggregation showed that the purified preps contained >90% of the expected MW species. A reverse phase chromatography method was established to monitor the “free” unconjugated ANP peptide levels in the final pools. Synthesized preps were verified to have less than 1% molar contamination of “free” ANP peptide. Mass spectroscopy techniques were used to assess the quality of the disulfide bond formation. The results of the mass spectroscopy indicated that the semi-synthetic constructs with longer linkers showed a small percentage of disulfide bond mixing. As the percentage with mixed disulfides was estimated to be very small fraction of the total, the synthesized preps were considered good for further analysis. Finally, Biacore analysis was performed to monitor binding of the semi-synthetic ANP-Fc fusion proteins to immobilized soluble human FcRn. This analysis was done to verify that the synthetic chemistry does not alter the FcRn binding capabilities of the Fc domain. No significant changes in FcRn binding were observed.

Once the quality of the semi-synthetic ANP-Fc fusion constructs was confirmed, their potency was tested in the human NPRA cGMP induction assay by two different laboratories, and the data processed using two different methods (Table 2: Labs 1 & 2 and Results 1 & 2). Results 1 from both Labs 1 and 2 were generated by interpreting the raw Relative Luminescence Units (RLU). Results 2 from Lab 1 were generated by comparing the RLU to a standard curve made up of known concentrations of cGMP in the same assay. The EC50 values generated in both analyses showed the same trends. One trend seen is that the dimer constructs are more potent in this assay than the respective monomer constructs. It is also clear that the ANP-Fc fusions with short (2 aa) linkers are at least 2-fold weaker relative to those with longer (11 aa) linkers.

TABLE 2
Semi-synthetic ANP-Fc fusions and cGMP Induction Response from Human NPRA Transfected Cells

| | | cGMP | cGMP | cGMP |
|---|---|---|---|---|
| Construct | | EC₅₀ (nM) | EC₅₀ (nM) | EC₅₀ (nM) |
| Number | Construct Formula | Lab 1 (Results 1) | Lab 1 (Results 2) | Lab 2 (Results 1) |
| 6 | 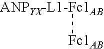 | 68 ± 23 (n = 4) | 150 ± 49 (n = 4) | NA |
| 7 | 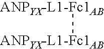 | 45 ± 22 (n = 3) | 114 ± 40 (n = 3) | NA |
| 8 | 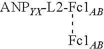 | 234 ± 199 (n = 2) | 329 ± 57 (n = 2) | 282 ± 123 (n = 3) |
| 9 | 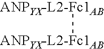 | 128 ± 161 (n = 4) | 118 ± 55 (n = 3) | 88 ± 21 (n = 3) |
| 10 | 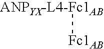 | 85 ± 16 (n = 2) | 327 ± 65 (n = 2) | 100 ± 39 (n = 4) |
| 11 | 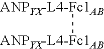 | 19 ± 4 (n = 2) | 68 ± 23 (n = 2) | 26 ± 11 (n = 4) |
| 12 | 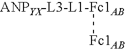 | 47 ± 6 (n = 4) | 158 ± 101 (n=4) | 77 ± 20 (n = 3) |
| 13 | 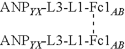 | 19 ± 11 (n = 4) | 66 ± 55 (n = 4) | 24 ± 2 (n = 3) |
| 14 | 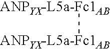 | NA | | 14 ± 1 (n = 2) |
| 15 | 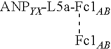 | NA | | 83 ± 51 (n = 3) |

In order to evaluate the correlation between linker length and potency, an ANP-Fc Fusion with a 16 aa linker (L5a) was generated (refer to Construct 14 and Construct 15). To evaluate even longer linkers, a recombinant 20 aa (L6) linked ANP-Fc fusion protein was generated. In both cases the composition of the longer linkers was kept consistent with the (GGS)x repeat pattern, where x is an integer from 0 to 16. Both the monomeric and dimeric ANP-Fc constructs with a 16 amino acid linker (L5a), shown in Table 2, demonstrated enhanced in vitro activity over the 11 amino acid linker (L4) constructs. To confirm that the semi-synthetic ANP-Fc fusion proteins generated were identical to recombinantly produced ANP-Fc fusions, recombinant constructs that mimic Construct 11 were also made as both IgG1 and IgG2 Fc fusions.

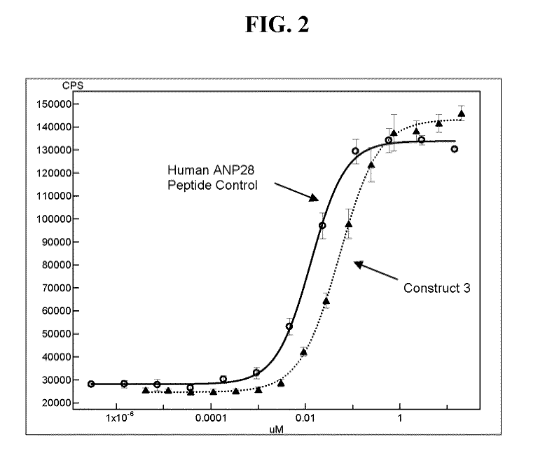

Human ANP28 peptide was assayed for cGMP production in species specific NPRA transfected 293 or 293T cells. Values for the concentration of fusion protein resulting in half-maximal stimulation of cGMP are listed in Table 3. Dose response curves for cGMP production stimulated by human ANP28 peptide in rat NPRA (rNPRA) expressing 293T cells and human NPRA (hNPRA) and canine NPRA (caNPRA) expressing 293 cells are shown in FIG. 2.

TABLE 3
Assay results for cGMP production in species
specific NPRA transfected 293 or 293T cells.

| Ligand | Assay Type | Mean EC50 nM | StdDev | N |
|---|---|---|---|---|
| ANP | cGMP-hNPRA 293 | 0.82 | 0.51 | 15 |
| ANP | cGMP-rNPRA 293 | 0.33 | 0.06 | 2 |
| ANP | cGMP-hNPRA 293 | 1.00 | 0.57 | 16 |
| ANP | cGMP-rNPRA 293 | 0.67 | 0.19 | 4 |
| ANP | cGMP-caNPRA 293 | 1.34 | 1.43 | 3 |

### Example 3

### Recombinant ANP Fusion Proteins

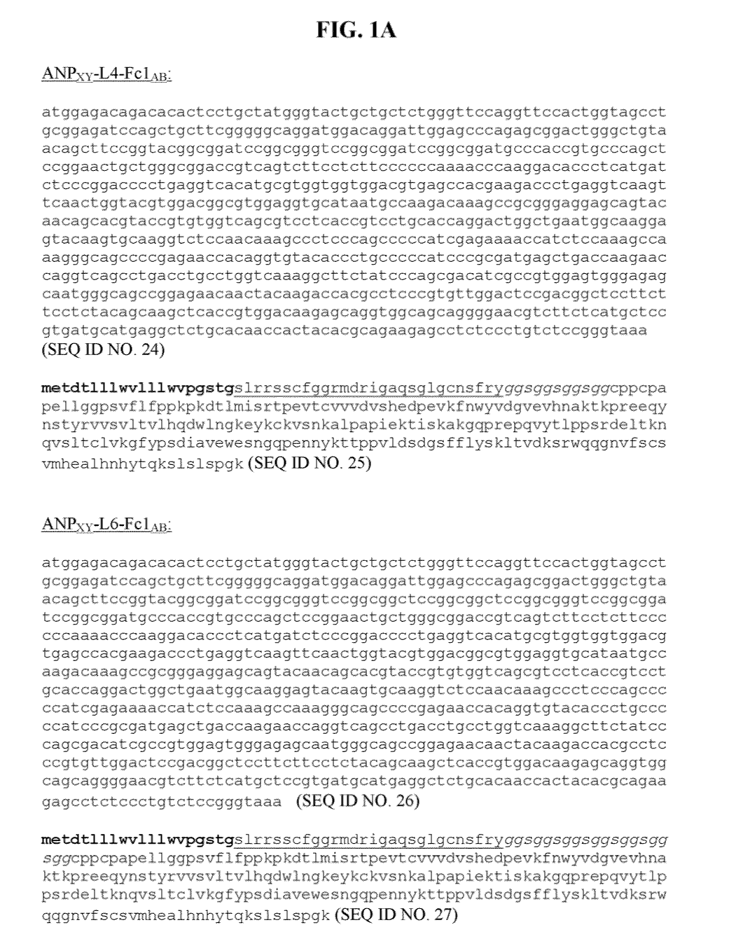

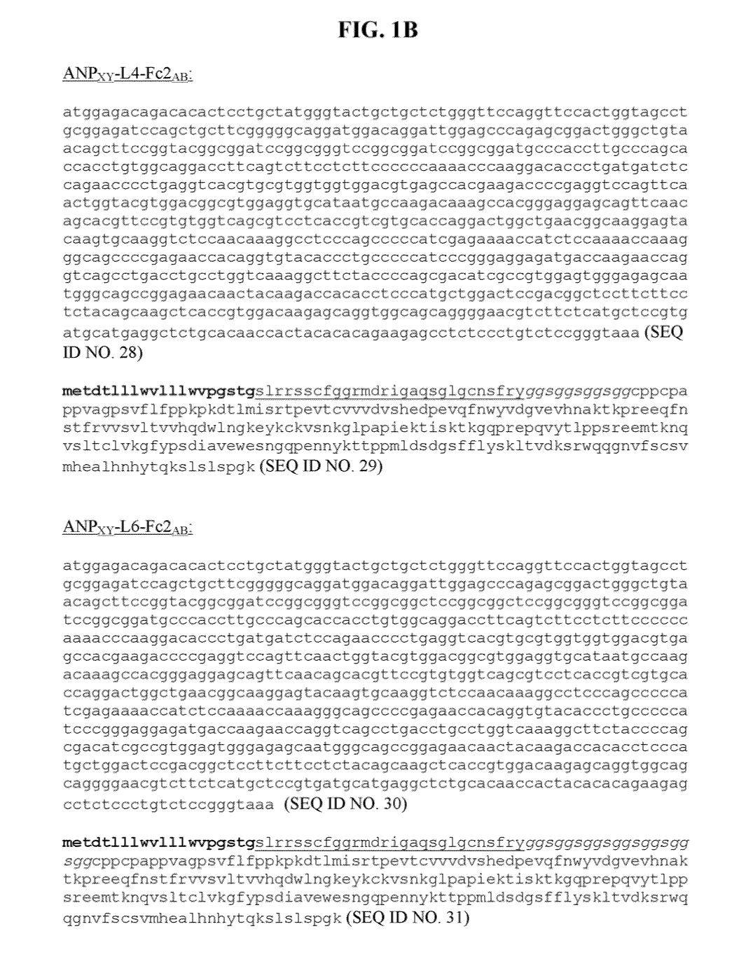

A production platform for the rapid generation of recombinant ANP-Fc fusion proteins was generated. The process starts with “base” Fc fusion vectors that allows DNA cassettes to be rapidly and seamlessly inserted onto the N-terminus of either IgG1 or IgG2 Fc. The Fc fusions to both IgG1 and IgG2 isotypes were generated in such a way that the hinge region is chopped down to the same CPPCP hinge residues thus ensuring that the linker extension and ANP fusion would be equally extended on the two isotypes. The respective DNA and protein sequences of the four recombinant ANP-Fc fusion proteins generated are represented by SEQ ID NOs: 24-31 (see, e.g., FIG. 1A-B). These fusion proteins each comprise a N-terminal mouse IgG kappa light chain signal sequence METDTLLLWVPGSTG (SEQ ID NO: 32) that is cleaved off and not part of the final protein product. The bolded The ANP-Fc fusion constructs were initially produced using 1 L transient mammalian expression followed by affinity chromatography. This production process typically yields 1-3 mg/L of >90% pure protein (by SDS-PAGE). To meet the increasing protein demands, two other mammalian expression technologies, large-scale (20 L) transient transfection and stable pool generation, were both successfully implemented. Both of these methodologies yielded at least 3-fold more protein per liter than the initial 1 L transient transfection. Overall these processes allowed for the production of more than 50 mg of each recombinant construct to be used for in vitro and in vivo analysis. The predicted MW of the non-reduced ANP-Fc fusion is 57.5 kDa, while the predicted MW of the reduced ANP-Fc fusion is 28.7 kDa.

To complement the recombinant production platform a characterization platform was put in place to ensure the quality of the ANP-Fc fusions being tested. As hANP28 has such high potency (see Table 4), and its sequence contains many critical amino acids and a disulfide necessary for functionality, mass spectroscopy (intact mass and tryptic digests) was utilized to monitor both “free” hANP28 peptide concentrations and cleavage products in the lots generated. A mass spectroscopy Endo-LysC digest protocol was also implemented to evaluate the N-terminal end of the ANP-Fc fusion peptides. Only lots with >90% intact N-termini were selected for further analysis. The level of aggregation was tested by sedimentation velocity experiments in an analytical ultracentrifuge where the lots were found to contain >85% monomer.

TABLE 4
Recombinant ANP-Fc fusions and cGMP Induction
Response Generated on Various NPRA Cell Types

| | | cGMP | cGMP | cGMP | cGMP |
|---|---|---|---|---|---|
| Construct | Construct | EC50 (nM) | EC50 (nM) | EC50 (nM) | EC50 (nM) |
| Number | Formula^{1,2} | Human NPRA | Rat NPRA | Dog NPRA | Monkey NPRA |
| 1 | ANP_{XY} | 1 +/− 0.5 | 0.7 +/− 0.2 | 1.3 +/− 1.4 | 0.5 +/− 0.2 |
| | | (n = 32) | (n = 4) | (n = 3) | (n = 3) |
| 2 | ANP_{XY}-L4- | 41 +/− 20 | 55 +/− 33 | 31 +/− 16 | 27 +/− 18 |
| | Fc1_{AB} | (n = 6) | (n = 3) | (n = 3) | (n = 3) |
| 3 | ANP_{XY}-L6- | 10 +/− 10 | 25 +/− 12 | 11 +/− 11 | 12 +/− 8 |
| | Fc1_{AB} | (n = 12) | (n = 9) | (n = 9) | (n = 3) |
| 4 | ANP_{XY}-L4- | 27 +/− 17 | 57 +/− 29 | 46 +/− 23 | 18 +/− 10 |
| | Fc2_{AB} | (n = 5) | (n = 5) | (n = 3) | (n = 3) |
| 5 | ANP_{XY}-L6- | 6 +/− 3 | 12 +/− 9 | 6 +/− 2 | 5 +/− 3 |
| | Fc2_{AB} | (n = 3) | (n = 3) | (n = 3) | (n = 3) |

¹See, e.g., formulas 3 and 4
²Note that the Fc domains of two different chains will dimerize upon recombinant expression, thus forming a dimer of the ANP.

The recombinantly produced ANP-Fc fusion constructs were tested in NPRA cGMP induction assays. The recombinant ANP-Fc fusion proteins tested to date show good cross-reactivity in the rat, canine and monkey assays. The 11 aa linker (L4) recombinantly produced ANP-Fc fusion proteins were similar in potency to the semi-synthetic construct Construct 11 that they mimic. This indicates that the potency data generated with the semi-synthetic ANP-Fc fusions is representative of what would be seen from a recombinantly generated protein. As expected, the variation in Fc isotype (Fc1 vs. Fc2) does not have significant impact on potency. The most dramatic potency effects were seen when comparing the various linker lengths. The 20 aa linker (L6) recombinantly produced ANP-Fc fusion proteins are clearly ˜2-fold more potent than the corresponding 11 aa linker (L4) fusion proteins. The semi-synthetic 16 aa linker (L5) ANP-Fc fusion protein Construct 14 has a human NPRA cGMP EC50 of 14±1 nM (n=2). Taking into account the semi-synthetic and recombinant ANP-Fc fusion potency data there is a direct correlation of in vitro potency to the linker length with 20 aa>16 aa>11 aa>2 aa.

The selectivity of the recombinant ANP-Fc fusion proteins was assessed by analyzing their efficacy in NPRB expressing cell lines. The species cross-reactivity of the ANP-Fc fusion proteins was tested in rat, canine, and monkey NPRA expressing HEK293 cell lines. The ANP-Fc fusions tested have good cross-reactivity in the rat, canine, and monkey NPRA mediated cGMP assays (Table 4). The ANP-Fc fusion proteins have nearly equivalent dose response values when tested in human, dog and monkey expressing NPRA cells. These results are not surprising given the fact that human, dog, and monkey ANP28 are 100% homologous. The dose response values for rat NPRA, on the other hand are ˜2-fold weaker, likely due to the fact that rat ANP differs from human ANP by one amino acid. Wild-type rat ANP has an EC₅₀ slightly higher than human ANP when assayed in the human NPRA cGMP assay.

### Example 4

### In Vivo Pharmacokinetic Studies

In vivo pharmacokinetic studies were performed to further characterize the fusion proteins of the present invention.

Pharmacokinetic properties of the semi-synthetic 11 (L4) and 16 (L5) amino acid linked ANP-Fc fusion monomers (Construct 10 and Construct 14, respectively) and dimers (Construct 11 and Construct 15, respectively) was obtained using intravenous dosing into Wistar rats. Two different ELISAs were used to detect the protein. The ANP/Fc ELISA was used to detect intact ANP-Fc. The Fc/Fc ELISA was used to detect the Fc protein and therefore measure both intact ANP-Fc and Fc to study the possibility of ANP peptide degradation.

Both monomeric constructs (Construct 10 and Construct 14) possessed an increased Cₘₐₓ over that of the dimers (Construct 11 and Construct 15), although the half-lives for the two types of constructs was similar (Table 5). This resulted in AUC's for the monomers that were >5-fold greater than those of the corresponding dimers (Table 5). No significant difference was detected between the two linker lengths on their pharmacokinetics. In addition, it was found that the ANP/Fc and Fc/Fc detection methods provided significantly different results with the Fc/Fc ELISA providing elevated serum concentrations and prolonged half-lives. This data suggests that the ANP-Fc fusion may have partially degraded with time in vivo.

TABLE 5
Pharmacokinetic Parameters of Monomer and Dimer in Wistar Rats

| Construct | | | Cmax | t½ | AUC | Cl | Vz | MRT |
|---|---|---|---|---|---|---|---|---|
| Number | Route | Rat | (ng/ml) | (hr) | (hr*ng/ml) | (ml/hr/kg) | (ml/kg) | (hr) |
| 10 | i.v. | adult | 4250 | 3.9 | 13947 | 36 | 202 | 3.8 |
| 11 | i.v. | adult | 1280 | 2.8 | 2645 | 189 | 775 | 3.1 |
| 14 | i.v. | adult | 3834 | 4.5 | 14590 | 34 | 220 | 4.4 |
| 15 | i.v. | adult | 429* | 5.5 | 2525 | 198 | 1583 | 5.5 |
| 15 | p.o. | neonatal | 17.2 | 6.5 | 253 | 1799 | 18553 | 9.1 |

The pharamacokinetics of the recombinant ANP-Fc fusion candidates were assessed by running a single-dose rat PK study. Single bolus injections of ANP-Fc fusions at 1 mg/kg dose were given to Sprague Dawley rats via i.v. or s.c. injection. Vehicle control PBS injections were given to a control group of animals. Plasma samples were collected at various timepoints (0, 0.083, 0.5, 1, 4, 8, 24, 48, 72, 96, 120, and 168 hr). Animals were sampled for 1 week with a contingency group going up to 3 weeks. After sampling the animals were quarantined and the plasma samples with EDTA and Aprotinin are assayed in a sandwich ELISA where the ANP is captured with a monoclonal antibody and detected with an anti-human Fc antibody. The ELISA has a sensitivity of 1 ng/mL and may be used to detect ANP-Fc fusion proteins in rat plasma. This rat PK data permits the comparison of the in vivo half-life of the ANP-Fc fusion proteins to that of the native ligand, hANP28.

The PK data obtained using the intravenous bolus dosing of the 4 recombinant ANP-Fc fusions into the rats is shown in Table 6. The data obtained demonstrates that the terminal half-life (T½) values for the fusion proteins (˜11-17 hours) were significantly longer than the native human ANP. Native ANP is reported to have a 0.3 minute T½ in rat and 2-3 minute T½ in humans. The intravenous dosing PK data also showed that the ANP-Fc fusions showed low clearance and a moderate volume or distribution. From the intravenous dosing PK data none of the four recombinant ANP-Fc proteins could be differentiated.

TABLE 6
Listing the clearance (CLp), volume of distribution (Vss), half-
life (T½), and mean resonance time (MRT) data.

| Construct | CLp | Vss | T½ | MRT |
|---|---|---|---|---|
| Number | (mL/min/kg) | (L/kg) | (hr) | (hr) |
| 2 | 2.77 | 0.49 | 12.3 | 2.94 |
| 3 | 2.29 | 0.27 | 11.5 | 1.94 |
| 4 | 2.58 | 0.61 | 16.8 | 3.91 |
| 5 | 4.05 | 0.76 | 11.7 | 3.08 |

The PK data obtained from the subcutaneous dosing of the 4 recombinant ANP-Fc fusions into rats is shown in Table 7. The data obtained suggests that the fusions had a slow absorption phase that results in them having longer half-lives (˜18-23 hours). The subcutaneous dosing did however result in low plasma concentration levels. From the subcutaneous dosing PK data obtained no clear differentiation could be made between the four recombinant ANP-Fc fusion proteins.

TABLE 7
Listing of the maximum serum concentration obtained (Cmax),
the area under the curve (AUC), the half-life (T½),
and the percent bioavailability (% F) observed.

| Construct | Cmax | AUC | T½ | |
|---|---|---|---|---|
| Number | (ng/ml) | (ng · hr/mL) | (hr) | % F |
| 2 | 70.8 | 2680 | 18.2 | 22.2 |
| 3 | 71.1 | 2521 | 20.3 | 17.3 |
| 4 | 53.5 | 2671 | 23.1 | 20.6 |
| 5 | 44 | 2202 | 20.6 | 26.3 |

The presence of an Fc attached to therapeutically relevant ANP peptide may enable non-invasive delivery of the ANP-Fc protein via the FcRn transport pathway. Given that neonatal rats possess high expression levels of FcRn in the gut in the first 15 days of life they are provide a useful model to study FcRn transport. Therefore, to assess the efficiency of FcRn transport, neonatal rats are given a single oral dose of 0.5 mg/kg ANP-Fc dimer (Construct 14). The data suggests that the ANP-Fc protein is not taken up efficiently, and that there is significant breakdown of the protein in the gut as illustrated by the large signal given by the Fc-only ELISA.

### Example 5

### Fusion Proteins Demonstrate Increased Resistance to Proteolytic Degradation by NEP

Neutral Endopeptidase (NEP, aka Neprilysisn, CALLA neutral endopeptidse 24.11, EC 3.4.24.11) is a type II integral membrane protein. NEP is a zinc metallopeptidase involved in the degradation of several types of proteins, including natriuretic peptides, enkephalins, and substance P and is thought to be responsible for the removal of from 30 to 50% of ANP from the circulation. See, e.g., J. Kenny et al., Biochem. J. (1993) 291, 8348 (1993). An in vitro NEP stability assay has been developed for use as an in vitro tool to study this clearance mechanism.

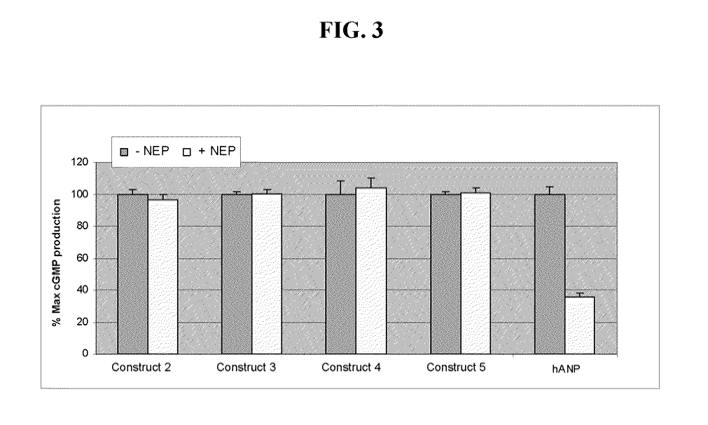

100 ng of recombinant human NEP (R&D Systems) was dissolved in cGMP assay buffer and added to the well of polypropylene microtiter plate containing 0.01 μM ANP (Sigma) or fusion protein. The plate was incubated for 60 minutes at room temperature. EDTA (Sigma) in calcium magnesium free PBS (Gibco) to 10 mM was added to each well to stop the reaction. The natriuretic peptide fusion protein induced cGMP assay was performed as described above. 15 μl of the NEP treated reaction mixture was added to the cell suspension and assayed for cGMP production. Data (mean of triplicate wells) indicated that none of the constructs tested show any significant loss in potency in NPRA mediated cGMP production (see, e.g., FIG. 3).

### Example 6

### Whole Cell Binding Demonstrated

The binding affinity (K_{d}) of the native ligand hANP for human NPRA expressed in whole cells was determined. Human NPRA transfected HEK293 cells were incubated with known concentrations of ¹²⁵I-labeled hANP28 at 4° C. for 2 hours in PEI coated 96-well glass fiber filter plates. Plates were washed with ice cold buffer by vacuum filtration and allowed to dry. Scintillant was added and the plates were counted on a TopCount Scintillation counter. Equilibrium model 350 (Michaelis-Menten equation [((BLmax*x)/(K_{d}+x))] is used to calculate the K_{d} value of 0.42 nM for hANP binding to NPRA.

The radioactive ligand whole cell receptor binding assay was used to compare the relative binding affinity of Construct 1 (hANP28) to that of the fusion proteins by performing a heterologous binding competition assay. NPRA transfectants were incubated with a either a fixed amount of ¹²⁵I-labeled ANP+/−excess unlabeled ANP or various concentrations of fusion protein at 4° C. for 2 hours in PEI coated 96-well glass fiber filter plates. Plates were washed using vacuum filtration and allowed to dry. Scintillant was added and the plates were counted on a TopCount Scintillation counter. Curves were fit using XLfit4.2 and IC₅₀ values (Table 8) were determined using the Dose Response One Site Model 205, [4 parameter fit: y=A+((B-A)/(1+((C/x)̂D)))]. The Kᵢ values (see e.g., Table 8) were calculated using the following formula, Kᵢ=IC₅₀/[1+([L]/K_{d})] where Kᵢ is the equilibrium dissociation constant for unlabelled ligand, IC₅₀ is the concentration causing 50% inhibition of binding, [L] is the concentration of radioligand, and K_{d} is the equilibrium dissociation constant for the radioligand. The candidates have 40 to 200-fold less affinity for NPRA than the native ligand. The rank affinity for each of the ligands tested is: Construct 1>Construct 5>Construct 3>Construct 4>Construct 2.

TABLE 8
IC₅₀, Kᵢ and relative affinity values for hANP28 and
each of the recombinant ANP-Fc fusion proteins.
Rank Affinity Determination

| | | | Relative |
|---|---|---|---|
| Ligand | IC₅₀ (nM) | Kᵢ (nM) | Affinity |
| Construct 1 | 5.5 ± 1 | 3.5 ± 2.6 | 1 |
| Construct 2 | 53.7 ± 1.6 | 24.1 ± 22.5 | 7 |
| Construct 3 | 11.9 ± 8 | 5.5 ± 3.1 | 2 |
| Construct 4 | 29.3 ± 37.2 | 12.3 ± 14.6 | 4 |
| Construct 5 | 4.3 ± 2.4 | 2 ± 0.9 | 1 |

Although the foregoing invention has been described in some detail by way of illustration and example for purposes of clarity of understanding, it will be readily apparent to those of skill in the art in light of the teachings of this invention that certain changes and modifications maybe made thereto without departing from the spirit or scope of the disclosure herein, including the appended embodiments.

1. A fusion protein comprising a natriuretic peptide and an antibody Fc domain, wherein said natriuretic peptide is conjugated to the Fc domain directly or through a linker.

2. The fusion protein of claim 1, wherein the fusion protein comprises the following formula:
X-Lₐ-F:F-Lₐ-X or X-Lₐ-F:F,
wherein,
X is a natriuretic peptide;
L is a linker comprising a amino acid residues;
a is an integer of at least 0;
: is a chemical association or crosslink; and
F is at least a portion of an immunoglobulin Fc domain comprising an FcRn binding site.

3. The fusion protein of any one of claim 1 or 2, wherein the natriuretic peptide is selected from the group consisting of ANP, BNP, Urodilatin, DNP or a biologically active sequence variant thereof.

4-9. (canceled)

10. The fusion protein of any one of claim 1 or 2, wherein the linker is 6 to 11 amino acids in length, 11 to 16 amino acids in length, 16 to 20 amino acids in length, 16 to 25 amino acids in length or 20 to 30 amino acids in length.

11-12. (canceled)

13. A fusion protein comprising at least one or more natriuretic peptides separated from each other by an antibody Fc domain, wherein said natriuretic peptides are conjugated to the Fc domain directly or through a linker.

14-28. (canceled)

29. A natriuretic fusion protein comprising any one of SEQ ID NOS. 8-11.

30. A natriuretic fusion protein comprising any one of SEQ ID NOS. 12-13.

31. An isolated polypeptide that exhibits at least 90% sequence identity with a polypeptide having a sequence selected from the group consisting of SEQ ID NO. 8, SEQ ID NO. 9, SEQ ID NO. 10 and SEQ ID NO. 11.

32. An isolated polypeptide that exhibits at least 90% sequence identity with a polypeptide having a sequence selected from the group consisting of SEQ ID NO. 12 and SEQ ID NO. 13.

33-36. (canceled)

37. An isolated nucleic acid molecule encoding a polypeptide comprising amino acid sequences selected from the group consisting of SEQ ID NO. 8, SEQ ID NO. 9, SEQ ID NO. 10 and SEQ ID NO. 11.

38. An isolated nucleic acid molecule encoding a polypeptide comprising amino acid sequences selected from the group consisting of SEQ ID NO. 12 and SEQ ID NO. 13.

39. A pharmaceutical composition comprising a fusion protein of claim 1.

40-42. (canceled)

43. A method of treating or ameliorating a disease or condition selected from the group consisting of: a condition characterized by an excessive level of extracellular fluid, a pathological condition in which activation of the NPRA receptor confers a therapeutic benefit, a disease associated with abnormal diruretic, natriuretic and vasodilatory activity, a disease in which it is desirable to induce naturesis, diuresis, vasodilation or to modulate the renin-angiotensin II and aldosterone systems, a pathological condition of the cardiovascular system selected from the group consisting of chronic heart failure (non-ischemic), reperfusion injury, left ventricular dysfunction (LVD), cardiac fibrosis, diastolic heart failure, and hypertrophic cardiomyopathy, a hypertensive disorder selected from the group consisting of hypertension, pulmonary hypertension, systolic hypertension and resistant hypertension, diabetic nephropathy, the method comprising administering to a subject in need thereof a therapeutically effective amount of the pharmaceutical composition of claim 39.

44-49. (canceled)

50. A conjugate comprising a natriuretic peptide and an antibody constant region, wherein the conjugate binds to a natriuretic peptide receptor.

51-61. (canceled)

62. An isolated nucleic acid comprising a polynucleotide sequence encoding a fusion polypeptide that comprises a natriuretic peptide and an antibody constant region.

63-65. (canceled)

66. An expression cassette comprising the nucleic acid of claim 62.

67. An isolated host cell transfected with the expression cassette of claim 66.

68. (canceled)

69. A method for recombinantly producing a fusion polypeptide comprising a natriuretic peptide and an antibody constant region, comprising the steps of: a. transfecting a host cell with the expression cassette of claim 66; and b. culturing the cell under the condition that are suitable for the cell to express the fusion polypeptide.

70. (canceled)

71. A composition comprising the conjugate of claim 50 and a pharmaceutically acceptable carrier.

72. A method for treating bacterial infection, pulmonary and bronchial diseases, renal failure, or heart failure, comprising the step of administering to a patient in need thereof an effective amount of the conjugate of claim 50.
